(19) United States
(12) Patent Application Publication
(10) Pub. No.: US 2026/0355285 A1
(43) Pub. Date: Nov. 12, 2026

(54) COMPOSITIONS FOR USE IN TREATING HEADACHE DISORDERS

(71) Applicant: AEON Biopharma, Inc., Irvine, CA (US)

(72) Inventor: Andrew Michael Blumenfeld, Del Mar, CA (US)

(21) Appl. No.: 19/127,948

(22) PCT Filed: Nov. 7, 2023
(86) PCT No.: PCT/US2023/078959

§ 371 Date: May 7, 2025

Related U.S. Application Data
(60) Provisional application No. 63/423,158, filed on Nov. 7, 2022.

(51) Int. Cl. A61K 38/26; A61K 38/48; A61P 25/00

(52) U.S. Cl. CPC A61K 38/26; A61K 38/4893; A61P 25/00; C12Y 304/24069

(57) ABSTRACT

Disclosed herein are compositions and methods for use in treating headache such as Migraine, for example Episodic or Chronic Migraine, including methods comprising GLP-1 agonist administration.

### CROSS REFERENCE TO RELATED APPLICATION

This application claims the benefit of, and priority to, U.S. Provisional Patent Application Ser. No. 63/423,158, filed on Nov. 7, 2022, the entire disclosure of which is hereby incorporated by reference in its entirety.

### FIELD

The present disclosure is directed to medicaments and methods for treating headache. In particular, the disclosure is directed to the use of GLP-1 agonists for treating different headache disorders.

### BACKGROUND

Headache can be diagnosed and classified based on the severity of the pain, the frequency of the pain, and whether there is an underlying cause. The different types of headache are tabulated in an international classification termed ICHD-3. All sensations in the head, including pain, are generally controlled by a nerve system called the trigeminal-occipital-cervical complex. This complex is a component of the neurological system that provides an important pain transmitting link from the cranial vasculature to the central nervous system. The nerves in the trigeminal complex provide sensory information from the major blood vessels regulating cerebral blood flow and from smaller blood vessels located within the pain-sensitive meninges.

Headaches are broadly classified as primary or secondary; primary headaches are headaches that are not caused by an underlying condition. Examples of primary headaches include migraine, tension-type headaches, and cluster headaches. Migraine is a chronic, debilitating neurological disease that produces severe pain and other associated symptoms. Migraines are unilateral, pulsating, moderate to severe headaches and typically last from 4 to 72 hours. Symptoms may also include nausea, vomiting, photophobia, and phonophobia.

Cluster headache is an extremely severe episodic headache and is characterized by “clusters” occurring for weeks or months. Individuals suffering from cluster headache may experience one to eight episodes per day, often right after falling asleep. Attacks often involve severe unilateral orbital pain lasting 15 minutes to 3 hours.

Tension-type headache is often a dull, bilateral, mild-to moderate-intensity pressure-pain. Infrequent tension-type headache is thought to be experienced by nearly everyone at some time.

Secondary headaches are headaches caused by some other condition or disorder. Examples of secondary headaches include headaches associated with trauma, headaches associated with substance withdrawal, headaches associated with sleep apnea, and headaches associated with raised intracranial pressure.

From a clinical perspective, tension-type is the most common type of headache; global prevalence of tension-type headache is approximately 40%. The global prevalence of migraine is much lower, but also known to be significantly more debilitating. Approximately 10% of individuals experiencing headaches globally suffer from migraine. In the United States, approximately 18% of women and 6% of men suffer from migraine. Frequent migraine, with headache occurring more than half the days of the month, is referred to as chronic migraine (CM). This makes up almost 10% of the migraine population.

Migraine is often associated with other conditions such as depression, anxiety, chronic pain syndromes and obesity. Cluster headache is often associated with benign pituitary tumors and sleep apnea. Sleep apnea is often linked to obesity. Headaches related to raised intracranial pressure are often associated with obesity.

The pathophysiology of migraine is based on the trigeminal-vascular system with the associated headache resulting from pain produced in intracranial blood vessels. In particular, the release of a neuropeptide, referred to as calcitonin gene-related peptide (“CGRP”), plays a central role in the pathophysiology of migraine. Migraine starts in the hypothalamus and this activates the brainstem, in particular the trigeminal system.

CGRP had been well-characterized. It was first discovered in 1983 in rats and then two forms were discovered in humans in 1984 and 1985. CGRP is a potent vasodilator and involved in pain. The cerebral blood vessels, contain a dense supply of CGRP-containing nerve fibers, which originate in the trigeminal ganglion. The CGRP-containing nerve fibers release CGRP during headaches such as migraine and cluster headache. CGRP levels are elevated during migraine. Inappropriate release of CGRP is a potential causative factor in several diseases, including migraine, type 2 diabetes, inflammation, and congestive heart failure.

Having recognized the link between CGRP release and migraine headache, researchers funded by several major pharmaceutical companies created small-molecule CGRP-receptor antagonists and anti-CGRP monoclonal antibodies as migraine treatments. Currently there are seven medications that are FDA approved to treat migraine by targeting CGRP.

CGRP is found in unmyelinated nerve endings termed C fibers and thinly myelinated nerve endings termed A delta fibers. The A delta fibers have CGRP receptors on their surface and released CGRP binds to these receptors creating a feedback loop that causes these nerves to keep firing throughout a migraine attack. Circulating CGRP levels are increased proportionate to increases in adipose tissue. CGRP is elevated in many different headache disorders including migraine, cluster, post traumatic headache and chronic paroxysmal hemicrania.

Among the types of headaches, migraine imposes a disproportionately large social and financial burden on patients, their families, and society. The Migraine Research Foundation estimates that U.S. employees take 113 million sick days per year because of migraines, creating an annual loss of $13 billion, and 53% of migraine patients reported that their severe headaches substantially impaired their daily activities. In the U.S., some 85% of migraine patients living with a partner reported substantial reduction in their ability to do household work, with 45% forced to miss social, family, and leisure activities; as much as 50% reported a negative effect on time spent with children and partners.

Thus, there is a need for an improved method of treating some headache disorders including CM and episodic migraine (EM).

### SUMMARY

Embodiments of the disclosure provide compositions and methods for effectively treating headaches including those that have not responded to other medications, by administering a GLP-1 agonist. Disclosed herein are compositions comprising at least one GLP-1 agonist, for example a peptide agonist, and the use thereof to treat headache, for example migraine, for example EM or CM, with reduced side effects and comparable or improved efficacy as compared to known methods.

In one aspect, the present disclosure provides methods for treating a migraine, a cluster headache, a post-traumatic headache, a headache attributed to sleep apnea, a headache attributed to raised intracranial pressure, or a refractory headache disorder, the methods comprising administering a GLP-1 agonist.

In embodiments, the GLP-1 agonist comprises a peptide. In embodiments, the GLP-1 agonist is administered subcutaneously, orally, intravenously, or inhaled.

Disclosed compositions and methods can further comprise neurotoxins, for example Clostridial neurotoxins, including botulinum toxins, and the use thereof to treat headache disorders in combination with a GLP-1 agonist, with reduced side effects and comparable or improved efficacy as compared to known methods.

Disclosed compositions can comprise solid or liquid dosage forms. Solid dosage forms can comprise, for example, powders, tablets, capsules, and the like. Liquid dosage forms can comprise, for example, sprays, injectables, and the like.

### BRIEF DESCRIPTION OF THE DRAWINGS

FIG. 1 shows the approximate location of several injection sites in various muscle regions disclosed herein.

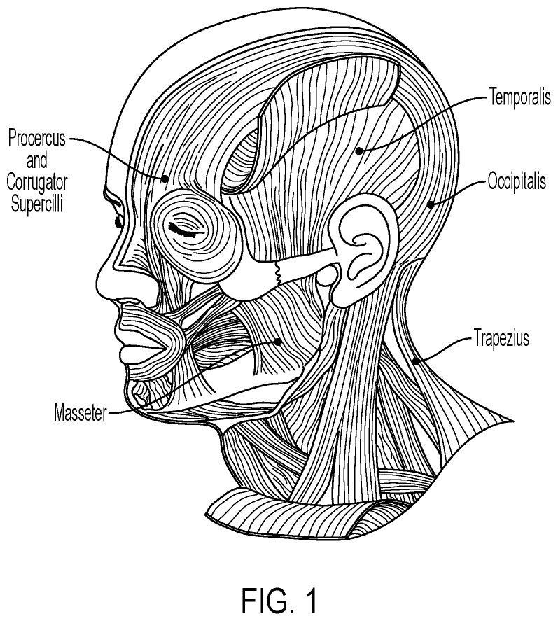

FIG. 2 shows the approximate location of several injection sites in various muscle regions disclosed herein.

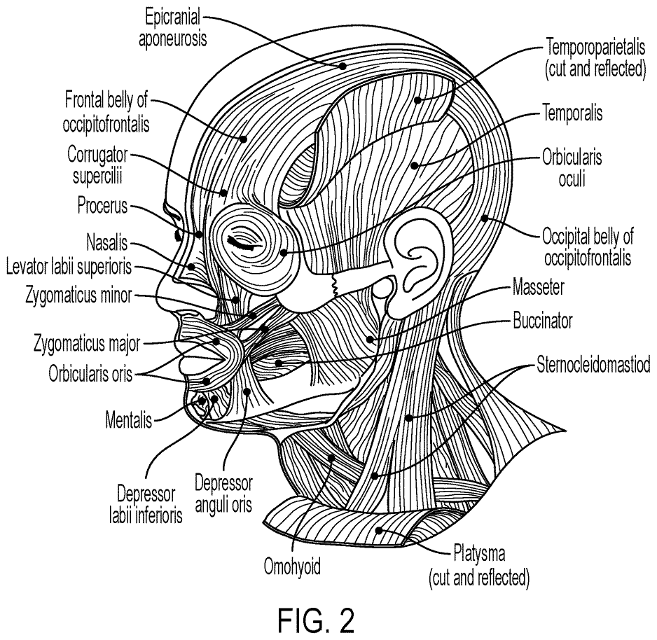

FIG. 3 shows the Sphenopalatine Ganglion.

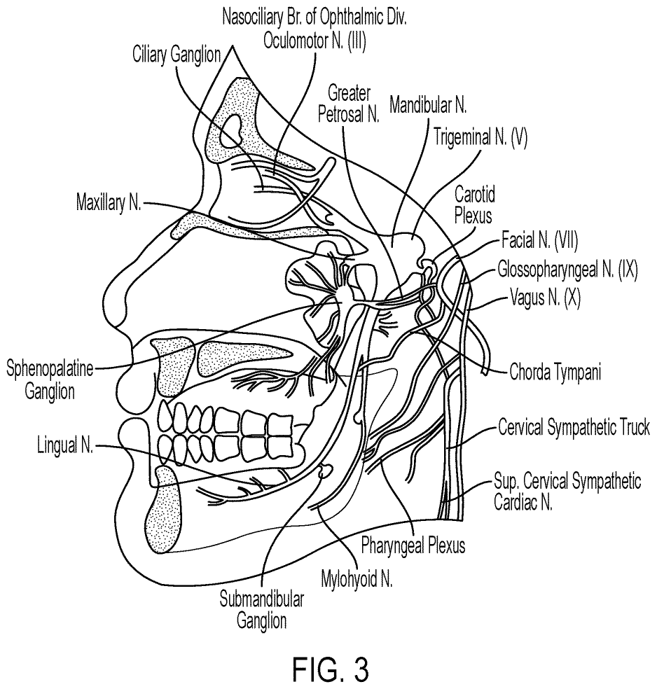

FIG. 4 shows the approximate location of several injection sites in various muscle regions as disclosed herein.

FIG. 5 shows the approximate anterior/lateral injection sites in various muscle regions used in a 150 Unit migraine treatment as described in Table 5 and the following paragraphs.

FIG. 6 shows the approximate posterior injection sites in various muscle regions used in a 150 Unit migraine treatment as described in Table 5 and the following paragraphs.

FIG. 7 shows the approximate anterior/lateral injection sites in various muscle regions used in a 195 Unit migraine treatment as described in Table 6 and the following paragraphs.

FIG. 8 shows the approximate posterior injection sites in various muscle regions used in a 195 Unit migraine treatment as described in Table 6 and the following paragraphs.

FIG. 9 shows anterior/lateral injection sites.

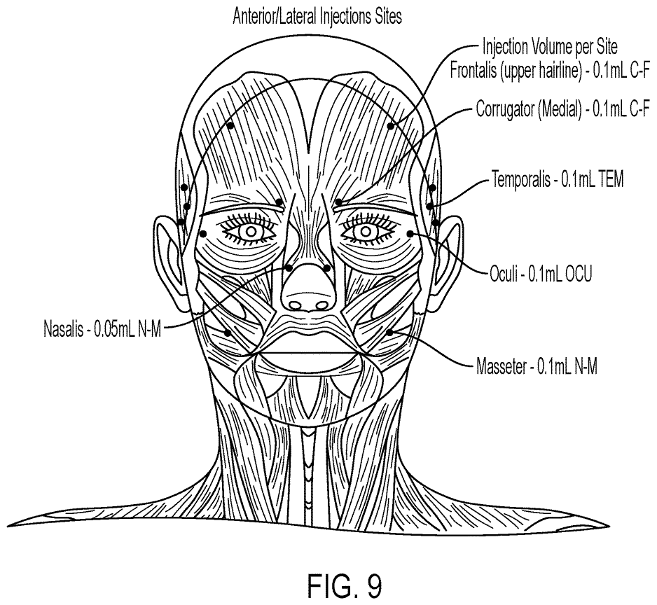

FIG. 10 shows posterior injection sites.

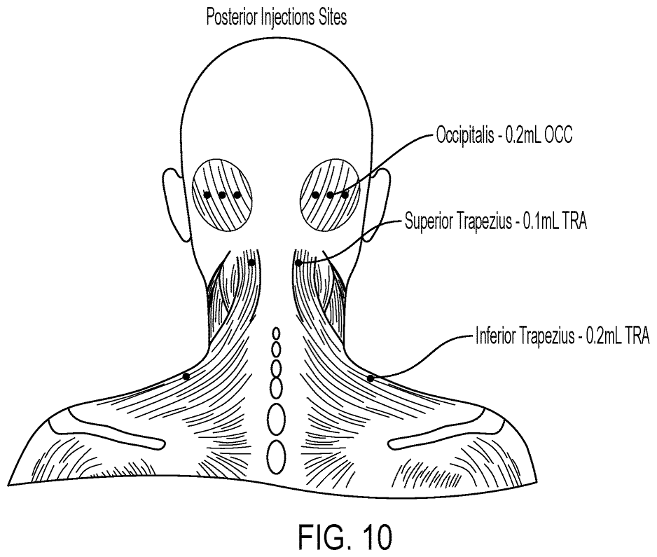

### DETAILED DESCRIPTION

The present disclosure is directed toward methods and compositions for treating headache patients, for example tension headache, CM, or EM patients. For example, in embodiments, a GLP-1 agonist is administered to the patient, thereby reducing the severity of symptoms associated with the headache such as pain, vision impairment, nausea, vomiting, sensitivity to light, smell or sound, depression, giddiness and/or anxiety. Disclosed embodiments can also lessen headache frequency.

Disclosed embodiments can further comprise administering a therapeutically effective amount of at least one neurotoxin to a nerve associated with at least one of the nasalis, orbicularis oculi, corrugator, procerus, masseter, occipitalis, temporalis, and trapezius muscle regions. In embodiments comprising injection into at least one of the nasalis, orbicularis oculi, corrugator, procerus, masseter, occipitalis, temporalis, and trapezius muscle regions, suitable compositions can comprise Clostridial neurotoxins, for example botulinum neurotoxins. Further embodiments can comprise injecting one of but not both of the frontalis and cervical paraspinal muscle regions. Further embodiments can comprise avoiding injecting at least one of or both of the frontalis and cervical paraspinal muscle regions.

Disclosed embodiments can comprise the avoidance of neurotoxin administration to specific areas. By avoiding administration to certain areas, the occurrence and/or severity of side effects can be reduced. In embodiments, the frontalis is not injected. In embodiments, this avoidance of injection to the frontalis prevents the development of ptosis in the patient. In embodiments, the cervical paraspinal muscles are not injected. In embodiments, this avoidance of injection to the cervical paraspinal muscles prevents the development of neck weakness in the patient.

Disclosed treatment modalities can prevent or alleviate symptoms of headache disorders, as well as both lessen the total neurotoxin dose administered to a patient and the number of administrations required. Longer duration of effect can also be provided by the disclosed methods. Disclosed combination treatments can provide a synergistic effect. Disclosed methods comprise treatment of migraine with fewer side effects.

Disclosed methods comprise fixed-site, fixed-dose methods targeting both sensory and motor effects. Disclosed methods provide for fewer injection sites as compared to current practices, as well as specialized treatments for EM and CM.

Disclosed embodiments include injection techniques that can minimize adverse effects, for example bruising, for example bruising associated with oculi injection. Disclosed embodiments include injection techniques comprising “injection threading,” an injection technique whereby the needle is inserted in a parallel, diagonal, or longitudinal direction into the muscle and toxin is gradually released as the needle is withdrawn. Disclosed embodiments can eliminate injection to the procerus, for example by utilizing a corrugator injection technique comprising injection into the corrugator parallel to the corrugator muscle utilizing injection threading to cover the procerus region.

### DEFINITIONS

“Administration,” or “to administer” means the step of giving (i.e. administering) a pharmaceutical composition or active ingredient to a subject. The pharmaceutical compositions disclosed herein can be administered via a number of appropriate routes, including oral, intramuscular, subdermal or subcutaneous routes of administration, such as by injection or use of an implant such as an extended-release implant. Intramuscular injections can be, for example, superficial or intermediate.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Fast-acting” as used herein refers to a botulinum toxin that produces effects in the patient more rapidly than those produced by, for example, a botulinum neurotoxin type A such as BOTOX®. For example, the effects of a fast-acting botulinum toxin (such as botulinum type E) can be produced within 36 hours.

“Fast-recovery” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as sweat glands in the treatment of hyperhidrosis. However, the usual duration of an intramuscular injection of a botulinum neurotoxin type A is typically about 3 to 4 months.

“GLP-1”, abbreviated for Glucagon-like peptide 1, as used herein encompasses any member of the Glucagon-like peptide family, including any analogs.

“GLP-1 agonist” refers to any molecule that exhibits any one or more of the following characteristics: (a) bind to GLP-1 receptors; (b) activation of GLP-1 receptor(s). Glucagon-like peptide-1 (GLP-1) agonists (also known as GLP-1 receptor agonists, incretin mimetics, or GLP-1 analogs) represent a class of medications currently used to treat type 2 diabetes mellitus and obesity in adults. Examples of drugs in this class include EXENATIDE®, LIXISENATIDE®, LIRAGLUTIDE®, ALBIGLUTIDE®, DULAGLUTIDE®, TIRZEPATIDE®, AND SEMAGLUTIDE®.

“Headache disorder” means any of the exemplar headaches disclosed herein or known in the art.

“Neurotoxin” or “neuromodulator” means a biologically active molecule with a specific affinity for a neuronal cell surface receptor. Neurotoxin includes Clostridial toxins both as pure toxins and as complexed with one to more non-toxin, toxin-associated proteins.

“Patient” means a human or non-human subject receiving medical or veterinary care.

“Peripherally administering” or “peripheral administration” as used herein encompasses oral, sublingual, buccal, rectal, via inhalation, intravenous, intra-arterial, intramuscular, intradermal, subdermal, subcutaneous, transdermal, transmucosal, and vaginal administrations.

“Pharmaceutical composition” means a formulation comprising an active agent. The word “formulation” means that there is at least one additional ingredient (such as, for example and not limited to, an albumin [such as a human serum albumin or a recombinant human albumin] and/or sodium chloride) in the pharmaceutical composition in addition to the active agent. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free.

“Therapeutic formulation” means a formulation that can be used to treat and thereby alleviate a disorder or a disease and/or symptom associated thereof.

“Therapeutically effective amount” means the level, amount or concentration of an agent (e.g. such as a GLP-1 agonist or Clostridial toxin or pharmaceutical composition comprising a GLP-1 agonist or Clostridial toxin or both) needed to treat a disease, disorder or condition without causing significant negative or adverse side effects.

“Treat,” “treating,” or “treatment” means an alleviation or a reduction (which includes some reduction, a significant reduction a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived symptom, disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®)).

GLP-1 is a member of the Glucagon-like family of peptides. GLP-1 agonists have been disclosed in methods for treatments of several disorders or diseases, including obesity and diabetes. The present disclosure is based in part, upon the discovery that peripheral administration of a GLP-1 agonist results in beneficial effects in patients with headache disorders.

The mechanism for GLP-1 agonists to produce beneficial effects in headache disorders include the following:
a. Decreased body adipose tissue, leading to reduced levels of circulating CGRP is only one of the methods.
b. Other methods involve direct effects of GLP-1 on brain receptors that modulate the pathways involved in headache. The hypothalamus is involved in the initiation of a migraine tract and GLP-1 receptor agonists may modify this by decreasing brainstem activation from the Hypothalamus through solitary tract effects.

### Disclosed Compositions

Disclosed compositions can comprise at least one GLP-1 agonist. For example, disclosed compositions can comprise EXENATIDE®, LIXISENATIDE®, LIRAGLUTIDE®, ALBIGLUTIDE®, DULAGLUTIDE®, TIRZEPATIDE®, AND SEMAGLUTIDE®.

Such compositions can be formulated in any pharmaceutically acceptable formulation in any pharmaceutically acceptable form. The GLP-1 agonist can also be used in any pharmaceutically acceptable form supplied by any manufacturer.

Although the composition may only contain a single type of GLP-1 agonist, disclosed compositions can include two or more types of GLP-1 agonists, which can provide enhanced therapeutic effects of the disorders. Administering a single composition containing two different GLP-1 agonists can permit the effective concentration of each of them to be lower than if a single GLP-1 agonist is administered to the patient while still achieving the desired therapeutic effects.

While disclosed methods can comprise separate administration of a GLP-1 agonist and a neurotoxin, disclosed compositions can further comprise neurotoxins.

Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing a lyophilized powder for reconstituting in a suitable vehicle such as saline for injection.

In embodiments, the botulinum toxin is formulated in a solution containing saline and pasteurized HSA, which stabilizes the toxin and minimizes loss through non-specific adsorption. The solution can be sterile filtered (for example using a 0.2 μm filter), filled into individual vials, and then vacuum-dried to give a sterile lyophilized powder. In use, the powder can be reconstituted by, for example, the addition of sterile unpreserved normal saline (sodium chloride 0.9% for injection).

In an embodiment, botulinum type A is supplied in a sterile solution for injection with a 5-mL vial nominal concentration of 20 ng/ml in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects of the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

Disclosed neurotoxin compositions can be injected into the patient, for example using a needle or a needleless device. In certain embodiments, the method comprises sub-dermally injecting the composition in the individual. For example, administering may comprise injecting the composition through a needle of no greater than about 30 gauge. In certain embodiments, the method comprises administering a composition comprising a botulinum toxin type A.

Administration of the disclosed compositions can be carried out by syringes, catheters, needles and other means for injecting. The injection can be performed on any area of the mammal's body that is in need of treatment, however disclosed embodiments contemplate neurotoxin injection into the patient's head and neck.

More than one injection and/or sites of injection may be necessary to achieve the desired result. Also, some injections, depending on the location to be injected, may require the use of fine, hollow, TEFLON®-coated needles. In certain embodiments, guided injection is employed, for example by electromyography, x-ray, ultrasound, or fluoroscopic guidance, or the like.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician.

### Methods of Use

Methods disclosed herein can comprise administration of a therapeutically effective amount of at least one GLP-1 agonist to a patient in order to prevent or alleviate the symptoms associated with headache. For example, disclosed methods can prevent or alleviate the occurrence of pain, nausea, vomiting, light sensitivity, sound sensitivity, and combinations thereof.

Further embodiments comprise administration of at least one neurotoxin. This combination can be administered as a single composition comprising the GLP-1 agonist and the neurotoxin, or the administration can comprise separate formulations. The separate formulations can be administered at the same treatment session or in successive treatment sessions.

In embodiments, disclosed methods comprise administering a therapeutically effective amount of at least one neurotoxin to a nerve associated with at least two of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with at least three of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with at least four of the nasalis, orbicularis oculi, corrugator, procerus, masseter occipitalis, temporalis, and trapezius muscle regions. In embodiments, methods comprise administering a therapeutically effective amount of at least one neurotoxin to a nerve associated with at least five of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with at least six of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with at least seven of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with at least eight of the nasalis, orbicularis oculi, corrugator, procerus, masseter, occipitalis, temporalis, and trapezius muscle regions. In embodiments, methods comprise administering a therapeutically effective amount of at least one neurotoxin to a nerve associated with the nasalis, orbicularis oculi, corrugator, procerus, masseter, occipitalis, temporalis, and trapezius muscle regions. Further embodiments can comprise injecting one of but not both of the frontalis and cervical paraspinal muscle regions. Further embodiments can comprise avoiding injecting at least one of or both of the frontalis and cervical paraspinal muscle regions.

In embodiments, methods comprise avoiding the administration of a therapeutically effective amount of at least one neurotoxin to at least one of the nasalis, orbicularis oculi, frontalis, corrugator, procerus, masseter, occipitalis, temporalis, trapezius and cervical paraspinal muscle regions. By avoiding certain administration locations, side effects can be lessened or eliminated.

For example, disclosed embodiments comprise administration to the nasalis, or the nasalis and corrugator, or the nasalis and corrugator and procerus, or the nasalis and corrugator and procerus and occipitalis, or the nasalis and corrugator and procerus and occipitalis and temporalis, or the nasalis and corrugator and procerus and occipitalis and temporalis and trapezius and orbicularis oculi, or the nasalis and corrugator and procerus and occipitalis and temporalis and trapezius, or the corrugator and procerus and occipitalis and temporalis and trapezius and masseter muscle regions, or the nasalis and corrugator and procerus and occipitalis and temporalis and trapezius and masseter muscle regions, or the nasalis and corrugator and procerus and occipitalis and temporalis and trapezius and masseter and orbicularis oculi muscle regions.

For example, disclosed embodiments comprise administration to the frontalis, or the frontalis and corrugator, or the frontalis and corrugator and occipitalis, or the frontalis and corrugator and occipitalis and temporalis, or the frontalis and corrugator and occipitalis and temporalis and trapezius, or the frontalis and corrugator and occipitalis and temporalis and trapezius and orbicularis oculi, or the frontalis and corrugator and occipitalis and temporalis and trapezius and orbicularis oculi and nasalis, or the frontalis and corrugator and occipitalis and temporalis and trapezius and orbicularis oculi and masseter muscle regions, or the frontalis, corrugator and occipitalis and temporalis and trapezius and orbicularis oculi and masseter and nasalis muscle regions.

Disclosed embodiments also comprise administration to the superior and inferior trapezius, the occipitalis, the temporalis, the corrugator, the orbicularis oculi, and the upper frontalis or the procerus or both. Disclosed embodiments also comprise administration to the superior and inferior trapezius, the occipitalis, the temporalis, the corrugator, the orbicularis oculi, the upper frontalis, and the nasalis or the masseter or both. Disclosed embodiments also comprise administration to the superior and inferior trapezius, the occipitalis, the temporalis, the upper frontalis, the corrugator, the orbicularis oculi, the nasalis and the masseter.

Further embodiments for treating headache comprises a neurotoxin injection protocol as described in Table 1:

TABLE 1

| | Sensory | Nr Sites/ | Total | Type of | | |
|---|---|---|---|---|---|---|
| | Targets | Side | Sites | Injection | Dose/Site | Total Dose |
| Trapezius | Grd Occipital | 2 | 4 | SD | 5U | 10U |
| | nerve and | (superior | | (Superior) | (Superior) | 20U |
| | Suprascapular | and | | IM | 10U | |
| | | inferior) | | (Inferior) | (Inferior) | |
| Occipitalis | Greater & Lesser | 3 | 6 | IM | 10U | 60U |
| | occipital nerves | | | | | |
| Temporalis | Auricular-temporal | 3 | 6 | IM | 5U | 30U |
| | and Zygomatic- | (enterior | | | | |
| | temporal branches | to tragus) | | | | |
| Procerus | Supratrochlear | 1 | 1 | IM | 5U | 5U |
| | and Supra-Orbital | | | | | |
| Corrugator | Supratrochlear | 1 | 2 | IM | 5U | 10U |
| | and Supra-Orbital | | | | | |
| Oculi | Zygomatic- | 1 | 2 | IM | 5U | 10U |
| | temporal branch | | | | | |
| Total | | 11 | 21 | | | 145U |

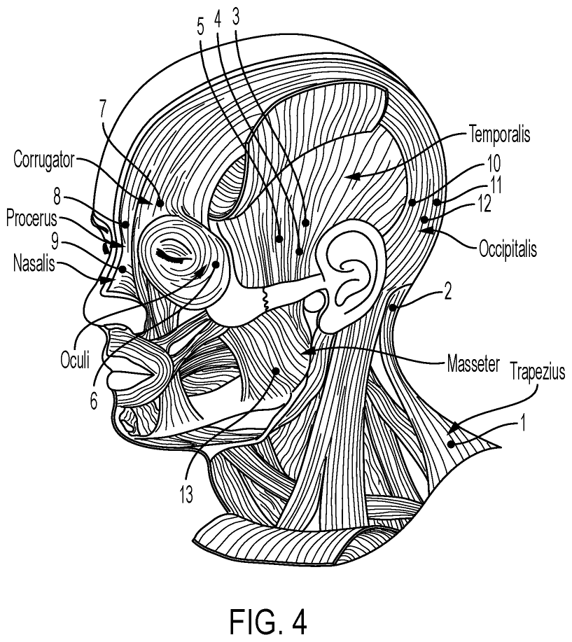

In Table 1, “Nr Sites/Side” means number of sites per side of head. “SD” means subdermal injection, “IM” means intramuscular injection, “SQ” means subcutaneous injection. The protocol of Table 1 can be adjusted based on treatment goals and results. For example, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 1 or superficial intramuscular injections as described on Table 2. The injections to the inferior trapezius can be intramuscular as described on Table 1 and Table 2.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 1, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is a about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 1, the number of injections to the temporalis per side (reference numerals 3, 4, and 5 in FIG. 4) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis, can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, or the like.

The total dosage to the temporalis can be, for example, 15 units, 20 units, 25 units, 30 units, 35 units, 40 units, 45 units, 50 units, 55 units, 60 units, or the like, or between 15 and 60 units, between 20 and 55 units, between 25 and 50 units, between 20 and 40 units, between 25 and 35 units, or the like.

In the embodiments of Table 1, the number of injections to the procerus (reference numeral 8 in FIG. 4) can be 1, 2, 3, 4, 5, or the like, or between 1 and 5 injections, between 1 and 4 injections, between 1 and 3 injections, between 2 and 5 injections, between 2 and 4 injections, between 1 and 3 injections, or the like.

The dosage per injection site to the procerus can be, for example, 3 units, 4 units, 5 units, 6 units, 7 units, 8 units, between 1 and 7 units, between 2 and 6 units, between 3 and 5 units, between 4 and 6 units, or the like.

The total dosage to the procerus can be, for example, 3 units, 4 units, 5 units, 6 units, 7 units, 8 units, between 1 and 7 units, between 2 and 6 units, between 3 and 5 units, between 4 and 6 units, or the like.

In the embodiments of Table 1, the number of injections to the corrugator per side (reference numeral 7 in FIG. 4) can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 1, the number of injections to the oculi per side (reference numeral 6 in FIG. 4) can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

Further embodiments for treating headache comprise a neurotoxin injection protocol as seen in Table 2:

TABLE 2

| | Sensory | Nr Sites/ | Total | Type of | | |
|---|---|---|---|---|---|---|
| | Targets | Side | Sites | Injection | Dose/Site | Total Dose |
| Trapezius | 3rd Occipital | 2 | 4 | SD | 5U | 10U |
| | nerve and | (superior | | (Superior) | (Superior) | 20U |
| | Suprascapular | and | | IM | 10U | |
| | | inferior) | | (Inferior) | (Inferior) | |
| Occipitalis | Greater & Lesser | 3 | 6 | IM | 10U | 60U |
| | occipital nerves | | | | | |
| Temporalis | Auricular-temporal | 3 | 6 | IM | 5U | 30U |
| | and Zygomatic- | (enterior | | | | |
| | temporal branches | to tragus) | | | | |
| Procerus | Supratrochlear | 1 | 1 | IM | 5U | 5U |
| | and Supra-Orbital | | | | | |
| Corrugator | Supratrochlear | 1 | 2 | IM | 5U | 10U |
| | end Supra-Orbital | | | | | |
| Oculi | Zygomatic- | 1 | 2 | IM | 5U | 10U |
| | temporal branch | | | | | |
| Total | | 11 | 21 | | | 145U |

In Table 2, “Nr Sites/Side” means number of sites per side of head. “Superficial IM” means superficial intramuscular injection, “IM” means intramuscular injection.

The protocol of Table 2 can be adjusted based on treatment goals and results. For example, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 1 or superficial intramuscular injections as described on Table 2. The injections to the inferior trapezius can be intramuscular as described on Table 1 and Table 2.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 2, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 2, the number of injections to the temporalis per side (reference numerals 3, 4, and 5 in FIG. 4) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, or the like.

The total dosage to the temporalis can be, for example, 15 units, 20 units, 25 units, 30 units, 35 units, 40 units, 45 units, 50 units, 55 units, 60 units, or the like, or between 15 and 60 units, between 20 and 55 units, between 25 and 50 units, between 20 and 40 units, between 25 and 35 units, or the like.

In the embodiments of Table 2, the number of injections to the procerus (reference numeral 8 in FIG. 4) can be 1, 2, 3, 4, 5, or the like, or between 1 and 5 injections, between 1 and 4 injections, between 1 and 3 injections, between 2 and 5 injections, between 2 and 4 injections, between 1 and 3 injections, or the like.

The dosage per injection site to the procerus can be, for example, 3 units, 4 units, 5 units, 6 units, 7 units, 8 units, between 1 and 7 units, between 2 and 6 units, between 3 and 5 units, between 4 and 6 units, or the like.

The total dosage to the procerus can be, for example, 3 units, 4 units, 5 units, 6 units, 7 units, 8 units, between 1 and 7 units, between 2 and 6 units, between 3 and 5 units, between 4 and 6 units, or the like.

In the embodiments of Table 2, the number of injections to the corrugator (reference numeral 7 in FIG. 4) per side can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 2, the number of injections to the oculi (reference numeral 6 in FIG. 4) per side can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

Further embodiments for treating headache comprise a neurotoxin injection protocol as seen in as seen in Table 3:

TABLE 3

| | Sensory | Nr Sites/ | Total | Type of | | |
|---|---|---|---|---|---|---|
| | Targets | Sice | Sites | Injection | Dose/Site | Total Dose |
| Trapezius | 3rd Occipital | 2 | 4 | SD | 5U | 10U |
| | nerve and | (superior | | (Superior) | (Superior) | 20U |
| | Suprascapular | and | | IM | 10U | |
| | | inferior) | | (Inferior) | (Inferior) | |
| Occipitalis | Greater & Lesser | 3 | 6 | IM | 10U | 60U |
| | occipital nerves | | | | | |
| Temporalis | Auricular-temporal | 3 | 6 | IM | 10U | 60U |
| | and Zygomatic- | (enterior | | | | |
| | temporal branches | to tragus) | | | | |
| Procerus | Supratrochlear | 1 | 1 | IM | 5U | 5U |
| | and Supra-Orbital | | | | | |
| Corrugator | Supratrochlear | 1 | 2 | IM | 5U | 10U |
| | and Supra-Orbital | | | | | |
| Oceli | Zygomatic- | 1 | 2 | IM | 5U | 10U |
| | temporal branch | | | | | |
| Nasalls | Nasocilary branch | 1 | 2 | SQ | 2.5U | 5U |
| | of infra-orbital | | | | | |
| Masseter | Trigeminal motor | 1 | 2 | IM | 5U | 10U |
| | division | | | | | |
| Total | | 13 | 25 | | | 190U |

In Table 3, “Nr Sites/Side” means number of sites per side of head. “SD” means subdermal injection, “IM” means intramuscular injection, “SQ” means subcutaneous injection. The protocol of Table 3 can be adjusted based on treatment goals and results. For example, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 3 or superficial intramuscular injections as described in Table 4. The injections to the inferior trapezius can be intramuscular as described in Table 3 and Table 4.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 3, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is a about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 3, the number of injections to the temporalis per side (reference numerals 3, 4, and 5 in FIG. 4) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis, can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, between 8 and 12 units, between 9 and 11 units, or the like.

The total dosage to the temporalis can be, for example, 15 units, 20 units, 25 units, 30 units, 35 units, 40 units, 45 units, 50 units, 55 units, 60 units, 65 units, 70 units, 75 units, 80 units, or the like, or between 20 and 80 units, between 25 and 75 units, between 30 and 70 units, between 35 and 65 units, between 50 and 70, between 55 and 65 units, or the like.

In the embodiments of Table 3, the number of injections to the procerus (reference numeral 8 in FIG. 4) can be 1, 2, 3, 4, 5, or the like, or between 1 and 5 injections, between 1 and 4 injections, between 1 and 3 injections, between 2 and 5 injections, between 2 and 4 injections, between 1 and 3 injections, or the like.

The dosage per injection site to the procerus can be, for example, 3 units, 4 units, 5 units, 6 units, 7 units, 8 units, between 1 and 7 units, between 2 and 6 units, between 3 and 5 units, between 4 and 6 units, or the like.

The total dosage to the procerus can be, for example, 3 units, 4 units, 5 units, 6 units, 7 units, 8 units, between 1 and 7 units, between 2 and 6 units, between 3 and 5 units, between 4 and 6 units, or the like.

In the embodiments of Table 3, the number of injections to the corrugator (reference numeral 7 in FIG. 4) per side can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 3, the number of injections to the oculi (reference numeral 6 in FIG. 4) per side can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

In the embodiments of Table 3, the number of subcutaneous injections per side to the nasalis (reference numeral 9 in FIG. 4) can be 1, 2, 3 or the like, or between 1 and 3 injections, or the like. The injections to the nasalis can be subcutaneous as described in Table 3 or superficial intramuscular as described in Table 4.

In the embodiments of Table 3, the total number of subcutaneous injections to the nasalis can be 1, 2, 3, 4, 5, 6, or the like, or between 1 and 6 injections, between 1 and 3 injections, between 2 and 4 injections, between 3 and 5, between 4 and 6, between 1 and 4, or the like.

The dosage per subcutaneous injection site to the nasalis can be, for example, 1 unit, 1.5 units 2 units, 2.5 units, 3 units, 3.5 units, 4 units, 4.5 units, 5 units, between 1 and 5 units, between 2 and 4 units, between 2 and 3 units, or the like.

The total subcutaneous dosage to the nasalis can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, between 5 and 10 units, between 5 and 8 units, or the like.

In the embodiments of Table 3, the number of injections per side to the masseter can be 1, 2, 3, 4, or the like, or between 1 and 4 injections, between 1 and 3 injections, between 2 and 4 injections, or the like.

In the embodiments of Table 3, the total number of injections to the masseter (reference numeral 13 in FIG. 4) can be 1, 2, 3, 4, 5, 6, 7, 8, or the like, or between 1 and 4 injections, between 1 and 3 injections, between 2 and 4 injections, between 4 and 6, between 4 and 8, or the like.

The dosage per injection site to the masseter can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the masseter can be, for example, 4 units, 5 units, 6 units, 7 units, 8 units, 9 units, 10 units, 11 units, 12 units, 13 units, 14 units, 15 units, 16 units, 17 units, 18 units, 19 units, 20 units, between 4 and 20 units, between 6 and 18 units, between 8 and 16 units, between 10 and 14 units, between 8 and 12 units, between 9 and 11 units, or the like.

Further embodiments for treating headache comprise a neurotoxin injection protocol as seen in as seen in Table 4:

TABLE 4

| | | | | | | | |
|---|---|---|---|---|---|---|---|
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |

[symbol]

indicates data missing or illegible when filed

In Table 4, “Nr Sites/Side” means number of sites per side of head. “Superficial IM” means superficial intramuscular injection, “IM” means intramuscular injection. The protocol of Table 4 can be adjusted based on treatment goals and results. For example, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 3 or superficial intramuscular injections as described on Table 4. The injections to the inferior trapezius can be intramuscular as described on Table 3 and Table 4.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 4, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is a about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 4, the number of injections to the temporalis per side (reference numerals 3, 4, and 5 in FIG. 4) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, between 8 and 12 units, between 9 and 11 units, or the like.

The total dosage to the temporalis can be, for example, 15 units, 20 units, 25 units, 30 units, 35 units, 40 units, 45 units, 50 units, 55 units, 60 units, 65 units, 70 units, 75 units, 80 units, or the like, or between 20 and 80 units, between 25 and 75 units, between 30 and 70 units, between 35 and 65 units, between 50 and 70, between 55 and 65 units, or the like.

In the embodiments of Table 4, the number of injections to the procerus (reference numeral 8 in FIG. 4) can be 1, 2, 3, 4, 5, or the like, or between 1 and 5 injections, between 1 and 4 injections, between 1 and 3 injections, between 2 and 5 injections, between 2 and 4 injections, between 1 and 3 injections, or the like.

The dosage per injection site to the procerus can be, for example, 3 units, 4 units, 5 units, 6 units, 7 units, 8 units, between 1 and 7 units, between 2 and 6 units, between 3 and 5 units, between 4 and 6 units, or the like.

The total dosage to the procerus can be, for example, 3 units, 4 units, 5 units, 6 units, 7 units, 8 units, between 1 and 7 units, between 2 and 6 units, between 3 and 5 units, between 4 and 6 units, or the like.

In the embodiments of Table 4, the number of injections to the corrugator (reference numeral 7 in FIG. 4) per side can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 4, the number of injections to the oculi (reference numeral 6 in FIG. 4) per side can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2 units, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

In the embodiments of Table 4, the number of superficial IM injections per side to the nasalis (reference numeral 9 in FIG. 4) can be 1, 2, 3 or the like, or between 1 and 3 injections, or the like. The injections to the nasalis can be subcutaneous as described in Table 3 or superficial intramuscular as described in Table 4.

In the embodiments of Table 4, the total number of superficial IM injections to the nasalis can be 1, 2, 3, 4, 5, 6, or the like, or between 1 and 6 injections, between 1 and 3 injections, between 2 and 4 injections, between 3 and 5, between 4 and 6, between 1 and 4, or the like.

The dosage per superficial IM injection site to the nasalis can be, for example, 1 unit, 1.5 units 2 units, 2.5 units, 3 units, 3.5 units, 4 units, 4.5 units, 5 units, between 1 and 5 units, between 2 and 4 units, between 2 and 3 units, or the like.

The total superficial IM dosage to the nasalis can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, between 5 and 10 units, between 5 and 8 units, or the like.

In the embodiments of Table 4, the number of injections (reference numeral 13 in FIG. 4) per side to the masseter can be 1, 2, 3, 4, or the like, or between 1 and 4 injections, between 1 and 3 injections, between 2 and 4 injections, or the like.

In the embodiments of Table 4, the total number of injections to the masseter can be 1, 2, 3, 4, 5, 6, 7, 8, or the like, or between 1 and 4 injections, between 1 and 3 injections, between 2 and 4 injections, between 4 and 6, between 4 and 8, or the like.

The dosage per injection site to the masseter can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the masseter can be, for example, 4 units, 5 units, 6 units, 7 units, 8 units, 9 units, 10 units, 11 units, 12 units, 13 units, 14 units, 15 units, 16 units, 17 units, 18 units, 19 units, 20 units, between 4 and 20 units, between 6 and 18 units, between 8 and 16 units, between 10 and 14 units, between 8 and 12 units, between 9 and 11 units, or the like.

Further embodiments for treating headache comprise a neurotoxin injection protocol as seen in as seen in Table 5:

| Targeted Nerves/ | Muscles to | Nr Sites/ | Total | Type of | | |
|---|---|---|---|---|---|---|
| Nerve Pathways | Inject | Side | Sites | Injection | Dose/Site | Total Dose |
| Supra-orbital | Upper | 1 | 2 | IM | 5U | 10U |
| | Frontalis | (at hairline) | | | | |
| Supratrochlear | Corrugator | 1 | 2 | IM | 5U | 10U |
| and Supra-Orbital | | | | | | |
| Zygomatic- | Oculi | 1 | 2 | IM | 5U | 10U |
| temporal branch | | | | | | |
| Auricular-temporal | Temporalis | 3 | 6 | IM | 5U | 30U |
| and Zygomatic- | | (anterior | | | | |
| temporal branches | | to tragus) | | | | |
| Greater & Lesser | Occipitalis | 3-2 | 6 | IM | 10U | 60U |
| occipital nerves | | | | | | |
| 3rd | Trapezius | 2 | 4 | Superficial | 5U | 10U |
| Occipital nerve | | (superior | | IM | (Superior) | 20U |
| and | | and | | (Superior) | 10U | |
| Suprascapular | | inferior) | | IM | (Inferior) | |
| | | | | (Inferior) | | |
| Total | | 11 | 22 | | | 150U |

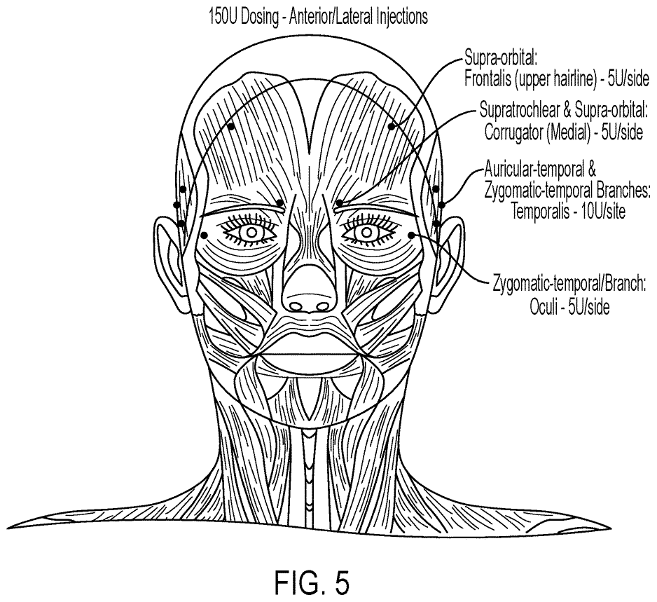

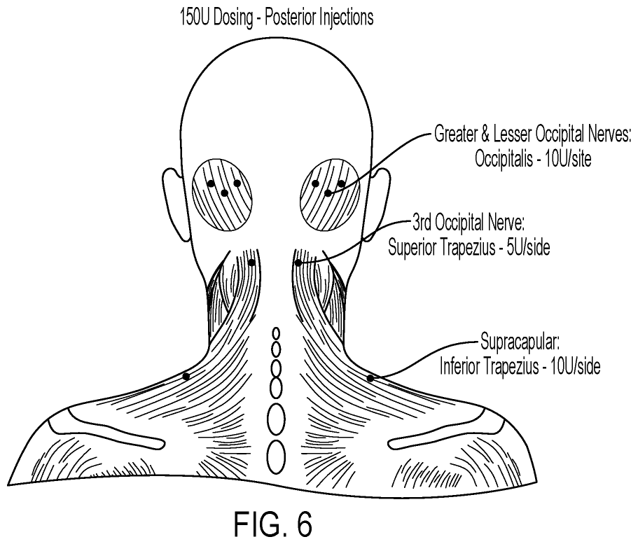

In Table 5, “Nr Sites/Side” means number of sites per side of head. “Superficial IM” means superficial intramuscular injection, “IM” means intramuscular injection. Injection locations associated with Table 5 are shown in FIGS. 5 and 6. In embodiments, injections directed toward the supraorbital nerve comprise upper frontalis injections at or near the hairline, comprising two lateral injections, one 5 unit injection in each side of the upper frontalis muscle for a total of 10 units. In some embodiments, the frontalis injection technique comprises starting at the limbic line where the forehead starts to angle backwards, injecting at a 45-degree angle, and angling away from the lower forehead. In embodiments, to target the frontalis injection the doctor determines the mid-pupillary line, visually moves up vertically to the hairline, punctures the skin at a 30 to 45 degree angle (to the skin), and makes a single injection at the hairline.

In embodiments, injections directed toward the supratrochlear and supraorbital nerves comprise injecting the corrugator muscles near the medial line, utilizing 2 parallel linear threading approach injections, with one 5 unit injection in each side of the medial corrugator muscle for a total of 10 units. In embodiments, the injection technique comprises inserting the needle at the medial edge of the corrugator muscle (at the vertical crease line) and parallel to the orbital ridge horizontal plane (parallel to the muscle above the eyebrow) and perpendicular to the corrugator skin line. The needle is slightly angled to the skin coming in on the medial edge and the needle is inserted ½″ while holding the muscle. The injector begins depressing the plunger as the needle is withdrawn towards the injection site and stops at the medial line. This “linear threading” approach allows for diffusion of the toxin into the area of the procerus and supratrochlear nerve while minimizing the number of injections for the patient. In embodiments, to visualize the corrugator muscle, the doctor asks the patient to close their eyes tightly to produce vertical lines caused by the corrugator contracting. The doctor then identifies the corrugator and stabilizes the muscle with the index finger and thumb. In embodiments, the skin is punctured across the medial portion of the corrugator, at a 90 degree angle to the vertical line rising from the inside edge of the eyebrow, and parallel to the corrugator muscle, with the doctor's index finger on the patient's orbital rim and his thumb above the eyebrow.

In embodiments, injections directed toward the nerves of the zygomatic-temporal branch comprise injection of the oculi area with one 5 unit injection on each side of the face to the lateral side of the oculi muscle for a total of 10 units. In embodiments, injections are made at a 45-degree angle to the face with only the bevel inserted, while avoiding any vascular structures that are visible. In embodiments, to target the oculi injection the doctor identifies a small vein in the area of the orbicularis oculi so that it can be avoided. To avoid bruising, the doctor administers the injection very superficially after the muscle is identified, and stabilized by the doctor by tensioning the skin manually. In embodiments, the injection is administered a finger breadth from the epicanthus or epicanthal line on the bony ridge of the orbit away from the eye, while avoiding the vein to minimize bruising. In embodiments, only the bevel of the needle is inserted below the skin, with the needle bevel facing up.

In embodiments, injections directed toward the auricular-temporal and zygomatic-temporal branches comprise injecting the anterior temporalis area with three 5 unit or 10 unit injections in each side of the anterior temporalis area, anterior to the tragus, for a total of 30 units or 60 units. For the most anterior of the three temporal injections, the injections are made at the hairline, angling the needle back towards the hairline of the temporal area to avoid the antra-temporal fascia and to minimize atrophy in that region. The other temporal injections may be behind the hairline, parallel to the tragus and injected 45 degrees to the skull. In embodiments, the first temporalis injection is made in line with the tragus one to two finger breadths above the apex of the ear. The second injection is made a finger or finger and a half breadth vertically above the first injection, and the third injection is made a finger breadth anterior to that, all three injections are made within the hairline.

In embodiments, injections directed to the greater and lesser occipital nerves comprise injecting the occipitalis area with three 10 unit intramuscular injections in each occipitalis muscle for a total of 60 units. The injection technique comprises inserting the needle parallel to the nuchal ridge. The injector starts to depress the plunger as the needle is withdrawn towards the injection site (a parallel linear threading approach to maximize spread) and aims to inject the first half of the dose in the lateral region and the second half of the dose in the medial region. The “linear threading” approach should allow for diffusion of the toxin into the occipitalis area and greater and lesser occipital nerves and may allow for less injections in the occipitalis, for example, two injections per side versus three injections per side. In embodiments, to target the occipital injection the doctor identifies the landmarks of the inion, the mastoid process, and the nuchal ridge. The injections are made just above the nuchal ridge, with the first injection made in the middle of these three landmarks, then the second injection is made more medially, and then the third injection is made more laterally along the nuchal ridge.

In embodiments, injections directed to the 3^{rd} occipital nerve and suprascapular nerve comprise injecting superficially into the superior and inferior trapezius areas with two intramuscular injections into each trapezius muscle, 5 units in the superior trapezius muscle and 10 units into the inferior trapezius muscle, for a total of 30 units. The injection technique for the superior trapezius injection comprises injecting upward away from the mid-neck toward the base of the skull at a 45-degree angle, angled toward the lower hairline, and parallel to the C2. The injector starts to depress the plunger as the needle is withdrawn towards the injection site (a parallel linear threading approach to maximize spread). For the inferior trapezius, the injection technique comprises injecting at a 45-degree angle at the top of the slope of the trapezius. The “linear threading” approach should allow for diffusion of the toxin into the superior trapezius area and 3^{rd} occipital nerve. In embodiments, the superior trapezius is injected medially below the skull base, after the doctor identifies the skull base, just over the cisterna magna. The injection is made to the superior trapezius just below the occipital ridge and about a finger breadth lateral to the ridge.

In embodiments, the inferior trapezius injection is made by locating the necklace line, then moving two finger-breadths laterally to the medial necklace line, identifying the muscle, and injecting away from the midline so that any diffusion carries the toxin away from the more vertically-oriented trapezius muscle fibers.

In certain embodiments, one or more of the muscles identified in Table 5 may not be injected, thereby reducing the total dosage. In certain embodiments, one or more of the muscles not identified in Table 5, such as the nasalis, the masseter, or the procerus may be injected, thereby increasing the total dosage. In certain embodiments, the total number of injection sites per muscle or per side shown on Table 5 may be increased or reduced, thereby increasing or decreasing the dosage per muscle or per side. The total dosage administered can be in the range of about 140 U-200 U.

In certain embodiments, the method comprises injecting the composition sub-dermally, subcutaneously, intramuscularly, or through superficial intramuscular injections, into the individual. For example, administering may comprise injecting the composition through a 27 gauge needle, 28 gauge needle, 29 gauge needle, 30 gauge needle, 31 gauge needle, 32 gauge needle, and/or 33 gauge needle. In certain embodiments, the method comprises administering a composition comprising a botulinum toxin type A.

In embodiments, the administration comprises using a 32 gauge needle. In some embodiments, the administration comprises using a ½ inch long 32 gauge needle. In embodiments, the administration comprises using a 30 gauge needle. In some embodiments, the administration comprises using a ½ inch long 30 gauge needle.

The protocol of Table 5 can be adjusted based on treatment goals and results. For example, the number of injections to the upper frontalis at the hairline per side can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the upper frontalis can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the upper frontalis can be intramuscular injections as described in Table 5.

The dosage per injection site to the upper frontalis can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the upper frontalis can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 5, the number of injections to the corrugator per side (reference numeral 7 in FIG. 4) can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 5, the number of injections to the oculi (reference numeral 6 in FIG. 4) per side can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

In embodiments, the oculi muscles are injected with the bevel of the needle facing up, toward the skin surface.

In the embodiments of Table 5, the number of injections to the temporalis anterior to the tragus (reference numerals 3, 4, and 5 in FIG. 4) per side can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis, can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, or the like.

The total dosage to the temporalis can be, for example, 15 units, 20 units, 25 units, 30 units, 35 units, 40 units, 45 units, 50 units, 55 units, 60 units, or the like, or between 15 and 60 units, between 20 and 55 units, between 25 and 50 units, between 20 and 40 units, between 25 and 35 units, or the like.

In the embodiments of Table 5, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 5, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 3 or superficial intramuscular injections as described on Table 4. The injections to the inferior trapezius can be intramuscular as described on Table 3 and Table 4.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

Further embodiments for treating headache comprise a neurotoxin injection protocol as seen in as seen in Table 6:

| Targeted Nerves/ | Muscles to | Nr Sites/ | Total | Type of | | |
|---|---|---|---|---|---|---|
| Nerve Pathways | Inject | Side | Sites | Injection | Dose/Site | Total Dose |
| Supra-orbital | Upper | 1 | 2 | IM | 5U | 10U |
| | Frontalis | (at hairline) | | | | |
| Supratrochlear | Corrugator | 1 | 2 | IM | 5U | 10U |
| and Supra-Orbital | | | | | | |
| Zygomatic- | Oculi | 1 | 2 | IM | 5U | 10U |
| temporal branch | | | | | | |
| Auricular-temporal | Temporalis | 3 | 6 | IM | 10U | 60U |
| and Zygomatic- | | (anterior | | | | |
| temporal branches | | to tragus) | | | | |
| Nasociliary branch | Nasalis | 1 | 2 | Superficial | 2.5U | 5U |
| of Infra-orbital | | | | IM | | |
| Trigeminal motor | Masseter | 1 | 2 | IM | | 10U |
| division | | | | | | |
| Greater & Lesser | Occipitalis | 3 | 6 | IM | 10U | 60U |
| occipital nerves | | | | | | |
| 3rd | Trapezius | 2 | 4 | Superficial | 5U | 10U |
| Occipital nerve | | (superior | | IM | (Superior) | 20U |
| and | | and | | (superior) | 10U | |
| Suprascapular | | inferior) | | IM | (Inferior) | |
| | | | | (inferior) | | |
| Total | | 13 | 26 | | | 195U |

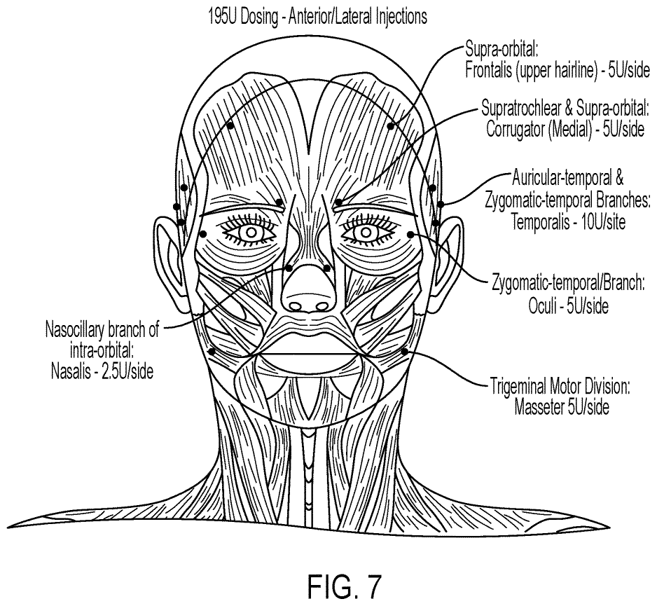

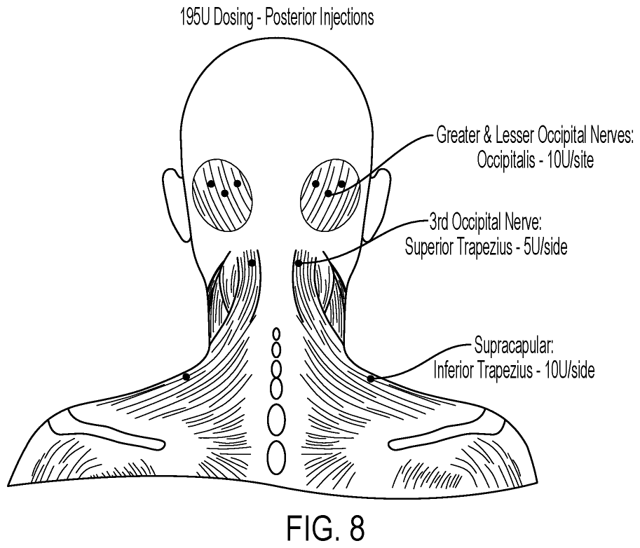

In Table 6, “Nr Sites/Side” means number of sites per side of head. “Superficial IM” means superficial intramuscular injection, “IM” means intramuscular injection. Injection locations associated with Table 6 are shown in FIGS. 7 and 8. In embodiments, injections directed toward the supraorbital nerve comprise upper frontalis injections at or near the hairline, comprising two lateral injections, one 5 unit injection in each side of the upper frontalis muscle for a total of 10 units. In some embodiments, the frontalis injection technique comprises starting at the limbic line where the forehead starts to angle backwards, injecting at a 45-degree angle, and angling away from the lower forehead. In embodiments, to target the frontalis injection the doctor determines the mid-pupillary line, visually moves up vertically to the hairline, punctures the skin at a 30 to 45 degree angle (relative to the skin) and makes a single injection at the hairline.

In embodiments, injections directed toward the supratrochlear and supraorbital nerves comprise injecting the corrugator muscles near the medial line, utilizing 2 parallel linear threading approach injections, with one 5 unit injection in each side of the medial corrugator muscle for a total of 10 units.

In embodiments, the injection technique comprises inserting the needle at the medial edge of the corrugator muscle (at the vertical crease line) and parallel to the orbital ridge horizontal plane (parallel to the muscle above the eyebrow) and perpendicular to the corrugator skin line. The needle is slightly angled to the skin coming in on the medial edge and the needle is inserted ½″ while holding the muscle. The injector begins depressing the plunger as the needle is withdrawn towards the injection site and stops at the medial line. This “linear threading” approach allows for diffusion of the toxin into the area of the procerus and supratrochlear nerve while minimizing the number of injections for the patient. In embodiments, to visualize the corrugator muscle, the doctor asks the patient to close their eyes tightly to produce vertical lines caused by the corrugator contracting. The doctor then identifies the corrugator and stabilizes the muscle with the index finger and thumb. In embodiments, the skin is punctured across the medial portion of the corrugator, at a 90 degree angle to the vertical line rising from the inside edge of the eyebrow, and parallel to the corrugator muscle, with the doctor's index finger on the patient's orbital rim and the thumb above the eyebrow.

In embodiments, injections directed toward the nerves of the zygomatic-temporal branch comprise injection of the oculi area with one 5 unit injection on each side of the face for a total of 10 units. In embodiments, injections are made at a 45-degree angle to the face with only the bevel inserted, while avoiding any vascular structures that are visible. In embodiments, to target the oculi injection the doctor identifies a small vein in the area of the orbicularis oculi so that it can be avoided. To avoid bruising, the doctor administers the injection very superficially after the muscle is identified and stabilized by the doctor by tensioning the skin manually. In embodiments, the injection is administered a finger breadth from the epicanthus or epicanthal line on the bony ridge of the orbit away from the eye, while avoiding the vein to minimize bruising. In embodiments, only the bevel of the needle is inserted below the skin. In embodiments, the oculi muscles are injected with the bevel of the needle facing up, toward the skin surface.

In embodiments, injections directed toward the auricular-temporal and zygomatic-temporal branches comprise injecting the anterior temporalis area with three 5 unit or 10 unit injections in each side of the anterior temporalis area, anterior to the tragus, for a total of 30 units or 60 units. For the most anterior of the three temporal injections, the injections are made at the hairline, angling the needle back towards the hairline of the temporal area to avoid the antra-temporal fascia and to minimize atrophy in that region. The other temporal injections may be behind the hairline, parallel to the tragus and injected 45 degrees to the skull. In embodiments, the first temporalis injection is made in line with the tragus a finger breadth or two above the apex of the ear. The second injection is made a finger or finger and a half breadth vertically above the first injection, and the third injection is made a finger breadth anterior to the second, with all three injections within the hairline.

In embodiments, injections directed toward the nasociliary branch of the infra-orbital nerve comprise injecting the nasalis area with one 2.5 unit superficial intramuscular injection in each side of the nasalis muscle, for a total of 5 units. The injection technique comprises inserting the needle at a 45-degree angle to the nose and inserting only the bevel of the needle. In embodiments, the doctor instructs the patient to “scrunch” their nose and eyes to identify the nasalis muscle, then the muscle is injected parallel to the bone away from the eye at a 90-degree angle to the skin, at a minimal depth.

In embodiments, injections directed toward the nerves of the trigeminal motor division comprise injecting the masseter area with one 5 unit intramuscular injection in each masseter muscle, for a total of 10 units. The injection technique comprises inserting the needle at a 45-degree angle. In embodiments, to identify the masseter, the doctor visualizes a line between the tragus and the corner of the mouth to avoid the zygomaticus. From halfway across that line, the doctor visually draws a line down to the angle of the jaw, and has the patient clench their teeth then relax. Then the masseter muscle is palpated, and the injection made perpendicular to the skin into the muscle, avoiding the parotid gland at a 90 degree angle as the patient maintains the tightened muscle. In embodiments, the toxin is administered as the patient relaxes the muscle, one to two finger breadths above the jawline to avoid the submandibular gland.

In embodiments, injections directed to the greater and lesser occipital nerves comprise injecting the occipitalis area with three 10 unit intramuscular injections in each occipitalis muscle for a total of 60 units. The injection technique comprises inserting the needle parallel to the nuchal ridge. The injector starts to depress the plunger as the needle is withdrawn towards the injection site (a parallel linear threading approach to maximize spread) and aims to inject the first half of the dose in the lateral region and the second half of the dose in the medial region. The “linear threading” approach should allow for diffusion of the toxin into the occipitalis area and greater and lesser occipital nerves and may allow for less injections in the occipitalis, for example, two injections per side versus three injections per side. In embodiments, to target the occipital injection the doctor identifies the landmarks of the inion, the mastoid process, and the nuchal ridge. The injections are made just above the nuchal ridge, with the first injection made in the middle of these three landmarks, then the second injection is made more medially, and then the third injection is made more laterally along the nuchal ridge.

In embodiments, injections directed to the 3^{rd} occipital nerve and suprascapular nerve comprise injecting the superior and inferior trapezius areas with two intramuscular injections into each trapezius muscle, 5 units superficially in the superior trapezius muscle and 10 units into the inferior trapezius muscle, for a total of 30 units. The injection technique for the superior trapezius injection comprises injecting upward away from the mid-neck toward the base of the skull at a 45-degree angle, angled toward the lower hairline, and parallel to the C2. The injector starts to depress the plunger as the needle is withdrawn towards the injection site (a parallel linear threading approach to maximize spread). For the inferior trapezius, the injection technique comprises injecting at a 45-degree angle at the top of the slope of the trapezius. The “linear threading” approach should allow for diffusion of the toxin into the superior trapezius area and 3^{rd} occipital nerve. In embodiments, the superior trapezius is injected medially below the skull base, after the doctor identifies the skull base, just over the cisterna magna. The injection is made to the superior trapezius just below the occipital ridge and about a finger breadth lateral to the ridge.

In embodiments, the inferior trapezius injection is made by locating the necklace line, then moving two finger-breadths laterally to the medial necklace line, identifying the muscle, and injecting away from the midline so that any diffusion carries the toxin away from the more vertically-oriented trapezius muscle fibers.

In certain embodiments, one or more of the muscles identified in Table 6 may not be injected, thereby reducing the total dosage. In certain embodiments, one or more of the muscles not identified in Table 6, such as the procerus may be injected, thereby increasing the total dosage. In certain embodiments, the total number of injection sites per muscle or per side shown on Table 6 may be increased or reduced, thereby increasing or decreasing the dosage per muscle or per side. The total dosage administered can be in the range of about 140 U-200 U.

In certain embodiments, the method comprises injecting the composition sub-dermally, subcutaneously, intramuscularly, or through superficial intramuscular injections, into the individual. For example, administering may comprise injecting the composition through a 27 gauge needle, 28 gauge needle, 29 gauge needle, 30 gauge needle, 31 gauge needle, 32 gauge needle, and/or 33 gauge needle. In certain embodiments, the method comprises administering a composition comprising a botulinum toxin type A.

In embodiments, the administration comprises using a 32 gauge needle. In some embodiments, the administration comprises using a ½ inch long 32 gauge needle. In embodiments, the administration comprises using a 30 gauge needle. In some embodiments, the administration comprises using a ½ inch long 30 gauge needle.

The protocol of Table 6 can be adjusted based on treatment goals and results. For example, the number of injections to the upper frontalis at the hairline per side can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the upper frontalis can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the upper frontalis can be intramuscular injections as described in Table 5.

The dosage per injection site to the upper frontalis can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the upper frontalis can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In the embodiments of Table 5, the number of injections to the corrugator per side (reference numeral 7 in FIG. 4) can be 1, 2, 3, 4, or the like, or between 1 and 4 injections, between 1 and 3 injections, between 2 and 4 injections, or the like. The injections to the corrugator can be parallel to the muscle. The injections to the corrugator can be to multiple total sites, for example, 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the corrugator can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the corrugator can be, for example, 3 units, 4 units, 5 units, 6 units, 7 units, 8 units, 9 units, 10 units, 11 units, 12 units, 13 units, 14 units, 15 units, 16 units, between 3 and 16 units, between 3 and 14 units, between 3 and 12 units, between 4 and 16 units, between 5 and 15 units, between 6 and 14 units, between 7 and 13 units, between 8 and 12 units, between 9 and 11 units, or the like.

In the embodiments of Table 5, the number of injections to the oculi (reference numeral 6 in FIG. 4) per side can be 1, 2, 3, 4, or the like, or between 1 and 4 injections, between 1 and 3 injections, between 2 and 4 injections, or the like. The injections to the oculi can be to multiple total sites, for example 2 sites, 3 sites, 4 sites, 5 sites, 6 sites, or 7 sites, or 8 sites, or the like, or to between 2 and 8 sites, between 2 and 6 sites, between 2 and 4 sites, between 3 and 6 sites, between 3 and 5 sites, or the like.

The dosage per injection site to the oculi can be, for example, 1 unit, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the oculi can be, for example, 2, 3 units, 4 units, 5 units, 6 units, 7 units, 8 units, 9 units, 10 units, 11 units, 12 units, 13 units, 14 units, 15 units, 16 units, 17, 18, 19, and 20, between 2 and 20 units, between 4 and 18 units, between 6 and 16 units, between 8 and 14 units, between 8 and 12 units, between 9 and 11 units, or the like.

In embodiments, the oculi muscles are injected with the bevel of the needle facing up, toward the skin surface.

In the embodiments of Table 6, the number of injections to the temporalis per side (reference numerals 3, 4, and 5 in FIG. 4) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the temporalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the temporalis can be, for example, 2 units, 3 units, 4 units, 5 units, 10 units, 15 units, 20 units, between 2 and 10 units, between 4 and 8 units, between 4 and 6 units, between 5 and 20 units, between 5 and 15 units, between 5 and 10 units, between 10 and 15 units, between 8 and 12 units, between 9 and 11 units, or the like.

The total dosage to the temporalis can be, for example, 15 units, 20 units, 25 units, 30 units, 35 units, 40 units, 45 units, 50 units, 55 units, 60 units, 65 units, 70 units, 75 units, 80 units, or the like, or between 20 and 80 units, between 25 and 75 units, between 30 and 70 units, between 35 and 65 units, between 50 and 70, between 55 and 65 units, or the like.

In the embodiments of Table 4, the number of IM injections per side to the nasalis (reference numeral 9 in FIG. 4) can be 1, 2, 3 or the like, or between 1 and 3 injections, or the like. The injections to the nasalis can be subcutaneous as described in Table 3 or superficial intramuscular as described in Table 4.

In the embodiments of Table 4, the total number of IM injections to the nasalis can be 1, 2, 3, 4, 5, 6, or the like, or between 1 and 6 injections, between 1 and 3 injections, between 2 and 4 injections, between 3 and 5, between 4 and 6, between 1 and 4, or the like.

The dosage per IM injection site to the nasalis can be, for example, 1 unit, 1.5 units 2 units, 2.5 units, 3 units, 3.5 units, 4 units, 4.5 units, 5 units, between 1 and 5 units, between 2 and 4 units, between 2 and 3 units, or the like.

The total IM dosage to the nasalis can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, between 5 and 10 units, between 5 and 8 units, or the like.

In the embodiments of Table 6, the number of injections (reference numeral 13 in FIG. 4) per side to the masseter can be 1, 2, 3, 4, or the like, or between 1 and 4 injections, between 1 and 3 injections, between 2 and 4 injections, or the like.

In the embodiments of Table 6, the total number of injections to the masseter can be 1, 2, 3, 4, 5, 6, 7, 8, or the like, or between 1 and 4 injections, between 1 and 3 injections, between 2 and 4 injections, between 4 and 6, between 4 and 8, or the like.

The dosage per injection site to the masseter can be, for example, 2 units, 3 units, 4 units, 5 units, 6 units, 7 units, 8 units, 9 units, 10 units, between 2 and 10 units, between 3 and 9 units, between 4 and 8 units, between 4 and 6 units, or the like.

The total dosage to the masseter can be, for example, 4 units, 5 units, 6 units, 7 units, 8 units, 9 units, 10 units, 11 units, 12 units, 13 units, 14 units, 15 units, 16 units, 17 units, 18 units, 19 units, 20 units, between 4 and 20 units, between 6 and 18 units, between 8 and 16 units, between 10 and 14 units, between 8 and 12 units, between 9 and 11 units, or the like.

In the embodiments of Table 6, the number of injections to the occipitalis per side (reference numerals 10, 11, and 12 in FIG. 4; mid-point of nuchal ridge for the first injection, which is done above the ridge. In embodiments the second and third injections form an inverted triangle with the first, and there is a about 2-3 cm between each injection point) can be 1, 2, 3, 4, 5, 6, 7, or the like, or between 1 and 6 injections, between 1 and 5 injections, between 2 and 6 injections, between 3 and 6 injections, between 2 and 5 injections, between 2 and 4 injections, or the like. The injections to the occipitalis can be to multiple total sites, for example 4 sites, 5 sites, 6 sites, 7 sites, 8 sites, 9 sites, or the like, or to between 2 and 8 sites, between 2 and 7 sites, between 3 and 6 sites, between 3 and 5 sites, between 5 and 7 sites, between 4 and 8 sites, or the like.

The dosage per injection site to the occipitalis can be, for example, 5 units, 10 units, 15 units, 20 units, between 5 and 20 units, between 5 and 15 units, between 5 and 10 units, between 10 and 15 units, or the like.

The total dosage to the occipitalis can be, for example, 20 units, 25 units, 30 units, 35 units, 40 units, 45 units, 50 units, 55 units, 60 units, 65 units, 70 units, 75 units, 80 units, 85 units, or between 20 and 40 units, between 25 and 35 units, 35 and 80 units, between 35 and 65 units, between 45 and 70 units, between 50 and 80 units, between 40 and 85 units, between 50 and 70 units, between 55 and 65 units, between 45 and 70 units, between 55 and 75 units, between 65 and 80 units, or the like.

In the embodiments of Table 6, the number of injection locations to the trapezius per side (superior [reference numeral 2 in FIG. 4] and inferior [reference numeral 1 in FIG. 4]) can be 1, 2, 3, 4, 5, or the like, or between 1 or 2 injections per side, between 1 and 3 injections, between 1 and 4 injections, between 1 and 5 injections, between 2 and 4 injections, between 2 and 5 injections, or the like. The injections to the trapezius (superior and inferior) can be to multiple sites, for example 2 sites, 3 sites, 4 sites, 5 sites, 6 sites, or 7 sites, or 8 sites, or the like, or to between 2 and 8 sites, between 2 and 7 sites, between 3 and 6 sites, between 3 and 5 sites, or the like. The injections to the superior trapezius can be subdermal as described on Table 3 or superficial intramuscular injections as described in Table 4. The injections to the inferior trapezius can be intramuscular as described in Table 3 and Table 4.

The dosage per injection site to the trapezius (superior and inferior) can be, for example, 2 units, 5 units, 10 units, 15 units, 20 units, between 5 and 20 units, between 5 and 15 units, between 5 and 10 units, between 10 and 15 units, between 15 and 20 units, between 4 and 6 units, between 2 and 8 units, between 8 and 12 units, or the like.

The total dosage to the trapezius (superior and inferior) can be, for example, 5 units, 10 units, 15 units, 20 units, 25 units, 30 units, 35 units, 40 units, 45 units, 50 units, 55 units, 60 units, or between 5 and 40 units, between 5 and 35 units, between 5 and 30 units, between 10 and 40 units, between 10 and 35 units, between 10 and 30 units, between 15 and 40 units, between 15 and 35 units, between 15 and 30 units, between 20 and 40 units, between 20 and 30 units, between 25 and 40 units, between 25 and 35 units, between 30 and 60 units, between 35 and 55 units, or the like.

In embodiments, methods comprise avoiding administering a therapeutically effective amount of at least one neurotoxin to a nerve associated with at least one of the nasalis, orbicularis oculi, frontalis, corrugator, procerus, occipitalis, temporalis, trapezius, masseter and cervical paraspinal muscle regions. For example, disclosed embodiments comprise administration to the nasalis and orbicularis oculi and corrugator and procerus and occipitalis and temporalis and trapezius and masseter, but not the cervical paraspinal muscle region or the frontalis. Other disclosed embodiments comprise administration to the orbicularis oculi and corrugator and procerus and occipitalis and temporalis and trapezius, but not the cervical paraspinal muscle region or the frontalis or nasalis or masseter. Similarly, disclosed embodiments comprise administration to the corrugator and procerus and occipitalis and temporalis and trapezius, but not the frontalis muscle region, or administration to the corrugator and procerus and occipitalis and temporalis and trapezius, but not the frontalis muscle region or cervical paraspinal region.

In embodiments, the cervical paraspinal is not administered-to.

In embodiments, the frontalis is not administered-to.

In embodiments, the corrugator is not administered-to.

In embodiments, the masseter is not administered-to.

In embodiments, the procerus is not administered-to.

In embodiments, the occipitalis is not administered-to.

In embodiments, the temporalis is not administered-to.

In embodiments, the trapezius is not administered-to.

In embodiments, the orbicularis oculi is not administered-to.

In embodiments, the nasalis is not administered-to.

Further embodiments comprise methods of reducing the amount of neurotoxin used for treating a migraine comprising substituting a GLP-1 agonist for at least part of the neurotoxin dosage.

Further embodiments comprise methods of increasing the duration of a neurotoxin-based migraine treatment by supplementing the neurotoxin administration with a GLP-1 agonist.

### GLP-1 Agonist Dosages

The GLP-1 agonist can be administered in a therapeutically-effective amount. For example, the GLP-1 agonist can be administered at a dosage of between 0.5 μg and 50 μg, or between 5 μg and 45 μg, or between 10 μg and 30 μg, or between 15 μg and 25 μg, or the like. In embodiments, the dosage is 0.5 μg, 1 μg, 0.5 μg, 1.5 μg, 2 μg, 2.5 μg, 3 μg, 3.5 μg, 4 μg, 4.5 μg, 5 μg, 5.5 μg, 6 μg, 6.5 μg, 7 μg, 7.5 μg, 8 μg, 8.5 μg, 9 μg, 9.5 μg, 10 μg, or the like.

In embodiments, the dosage is not more than 0.5 μg, not more than 1 μg, not more than 0.5 μg, not more than 1.5 μg, not more than 2 μg, not more than 2.5 μg, not more than 3 μg, not more than 3.5 μg, not more than 4 μg, not more than 4.5 μg, not more than 5 μg, not more than 5.5 μg, not more than 6 μg, not more than 6.5 μg, not more than 7 μg, not more than 7.5 μg, not more than 8 μg, not more than 8.5 μg, not more than 9 μg, not more than 9.5 μg, not more than 10 μg, or the like.

In embodiments, the dosage is not less than 0.5 μg, not less than 1 μg, not less than 0.5 μg, not less than 1.5 μg, not less than 2 μg, not less than 2.5 μg, not less than 3 μg, not less than 3.5 μg, not less than 4 μg, not less than 4.5 μg, not less than 5 μg, not less than 5.5 μg, not less than 6 μg, not less than 6.5 μg, not less than 7 μg, not less than 7.5 μg, not less than 8 μg, not less than 8.5 μg, not less than 9 μg, not less than 9.5 μg, not less than 10 μg, or the like.

In further embodiments, the GLP-1 agonist can be administered at a dosage of between 0.5 mg and 50 mg, or between 5 mg and 45 mg, or between 10 mg and 30 mg, or between 15 mg and 25 mg, or the like. In embodiments, the dosage is 0.5 mg, 1 mg, 0.5 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, or the like.

In embodiments, the dosage is not more than 0.5 mg, not more than 1 mg, not more than 0.5 mg, not more than 1.5 mg, not more than 2 mg, not more than 2.5 mg, not more than 3 mg, not more than 3.5 μmg, not more than 4 mg, not more than 4.5 mg, not more than 5 mg, not more than 5.5 mg, not more than 6 mg, not more than 6.5 mg, not more than 7 mg, not more than 7.5 mg, not more than 8 mg, not more than 8.5 mg, not more than 9 mg, not more than 9.5 mg, not more than 10 mg, or the like.

In embodiments, the dosage is not less than 0.5 mg, not less than 1 mg, not less than 0.5 mg, not less than 1.5 mg, not less than 2 mg, not less than 2.5 mg, not less than 3 mg, not less than 3.5 mg, not less than 4 mg, not less than 4.5 mg, not less than 5 mg, not less than 5.5 mg, not less than 6 mg, not less than 6.5 mg, not less than 7 mg, not less than 7.5 mg, not less than 8 mg, not less than 8.5 mg, not less than 9 mg, not less than 9.5 mg, not less than 10 mg, or the like.

In embodiments, the GLP-1 agonist can be administered once or twice daily, once or twice or three times weekly, or the like. Disclosed embodiments can comprise the following administration:
a. DULAGLUTIDE®—once weekly;
b. ALBIGLUTIDE®—once weekly;
c. LIRAGLUTIDE®—once daily;
d. SEMAGLUTIDE®—one weekly subcutaneously, daily orally;
e. EXENATIDE® BID—twice daily;
f. EXENATIDE® QW—once weekly;
g. LIXISENATIDE®—once daily;
h. TIRZEPATIDE®—once weekly.

### Neurotoxin Dosages

In embodiments comprising administration of a neurotoxin, the neurotoxin can be administered in an amount of between about 10-3 U/kg and about 35 U/kg. In an embodiment, the neurotoxin is administered in an amount of between about 10-2 U/kg and about 25 U/kg. In another embodiment, the neurotoxin is administered in an amount of between about 10-1 U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 5 Units to about 200 Units of a neurotoxin, such as a botulinum type A, can be used and in another embodiment, from about 10 Units to about 100 Units of a neurotoxin, such as a botulinum type A, can be locally administered into a target tissue.

In embodiments, administration can comprise a total dose per treatment session of about 30 Units of a botulinum neurotoxin, or about 35 Units, or about 40 Units, or about 45 Units, or about 50 Units, or about 55 Units, or about 60 Units, or about 65 Units, or about 70 Units, or about 75 Units, or about 80 Units, or about 85 Units, or about 90 Units, or about 95 Units, or about 100 Units, or about 105 Units, or about 110 Units, or about 115 Units, or about 120 Units, or about 125 Units, or about 130 Units, or about 135 Units, or about 140 Units, or about 145 Units, or about 150 Units, or about 155 Units, or about 160 Units, or about 165 Units, or about 170 Units, or about 175 Units, or about 180 Units, or about 185 Units, or about 190 Units, or about 195 Units, or about 200 Units, or about 205 Units, or about 210 Units, or about 215 Units, or about 220 Units, or about 225 Units, or about 230 Units, or about 235 Units, or about 240 Units, or about 245 Units, or about 250 Units, or about 255 Units, or about 260 Units, or about 265 Units, or about 270 Units, or about 275 Units, or about 280 Units, or about 285 Units, or about 290 Units, or about 295 Units, or about 300 Units, or the like.

In embodiments, administration can comprise a total dose per treatment session of not less than 10 Units of a neurotoxin, for example botulinum type A neurotoxin, or not less than 15 Units, or not less than 20 Units, or not less than 25 Units, or not less than 30 Units, or not less than 35 Units, or not less than 40 Units, or not less than 45 Units, or not less than 50 Units, or not less than 55 Units, or not less than 60 Units, or not less than 65 Units, or not less than 70 Units, or not less than 75 Units, or not less than 80 Units, or not less than 85 Units, or not less than 90 Units, or not less than 95 Units, or not less than 100 Units, or not less than 105 Units, or not less than 110 Units, or not less than 115 Units, or not less than 120 Units, or not less than 125 Units, or not less than 130 Units, or not less than 135 Units, or not less than 140 Units, or not less than 145 Units, or not less than 150 Units, or not less than 155 Units, or not less than 160 Units, or not less than 165 Units, or not less than 170 Units, or not less than 175 Units, or not less than 180 Units, or not less than 185 Units, or not less than 190 Units, or not less than 195 Units, or not less than 200 Units, or not less than 205 Units, or not less than 210 Units, or not less than 215 Units, or not less than 220 Units, or not less than 225 Units, or not less than 230 Units, or not less than 235 Units, or not less than 240 Units, or not less than 245 Units, or not less than 250 Units, or not less than 255 Units, or not less than 260 Units, or not less than 265 Units, or not less than 270 Units, or not less than 275 Units, or not less than 280 Units, or not less than 285 Units, or not less than 290 Units, or not less than 295 Units, or not less than 300 Units, or the like.

In embodiments, administration can comprise a total dose per treatment session of not more than 10 Units of a neurotoxin, for example botulinum type A neurotoxin, or not more than 15 Units, or not more than 20 Units, or not more than 25 Units, or not more than 30 Units, or not more than 35 Units, or not more than 40 Units, or not more than 45 Units, or not more than 50 Units, or not more than 55 Units, or not more than 60 Units, or not more than 65 Units, or not more than 70 Units, or not more than 75 Units, or not more than 80 Units, or not more than 85 Units, or not more than 90 Units, or not more than 95 Units, or not more than 100 Units, or not more than 105 Units, or not more than 110 Units, or not more than 115 Units, or not more than 120 Units, or not more than 125 Units, or not more than 130 Units, or not more than 135 Units, or not more than 140 Units, or not more than 145 Units, or not more than 150 Units, or not more than 155 Units, or not more than 160 Units, or not more than 165 Units, or not more than 170 Units, or not more than 175 Units, or not more than 180 Units, or not more than 185 Units, or not more than 190 Units, or not more than 195 Units, or not more than 200 Units, or not more than 205 Units, or not more than 210 Units, or not more than 215 Units, or not more than 220 Units, or not more than 225 Units, or not more than 230 Units, or not more than 235 Units, or not more than 240 Units, or not more than 245 Units, or not more than 250 Units, or not more than 255 Units, or not more than 260 Units, or not more than 265 Units, or not more than 270 Units, or not more than 275 Units, or not more than 280 Units, or not more than 285 Units, or not more than 290 Units, or not more than 295 Units, or not more than 300 Units, or the like.

In embodiments, administration can comprise a total dose per year of not more than 400 Units of a neurotoxin, for example botulinum type A neurotoxin, or not more than 500 Units, or not more than 600 Units, or not more than 700 Units, or not more than 800 Units, or not more than 900 Units, or not more than 1000 Units, or the like.

In embodiments, neurotoxin administration, for example botulinum administration, to the nerve associated with at least one of the frontalis, corrugator, procerus, masseter occipitalis, temporalis, trapezius and cervical paraspinal muscle regions can comprise a total dose per treatment session of about 40 Units of a botulinum neurotoxin, or about 45 Units, or about 50 Units, or about 55 Units, or about 60 Units, or about 65 Units, or about 70 Units, or about 75 Units, or about 80 Units, or about 85 Units, or about 90 Units, or about 95 Units, or about 100 Units, or about 105 Units, or about 110 Units, or about 115 Units, or about 120 Units, or about 125 Units, or about 130 Units, or about 135 Units, or about 140 Units, or about 145 Units, or about 150 Units, or about 155 Units, or about 160 Units, or about 165 Units, or about 170 Units, or about 175 Units, or about 180 Units, or about 185 Units, or about 190 Units, or about 195 Units, or about 200 Units, or about 205 Units, or about 210 Units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Disclosed embodiments comprise treatments that can be repeated. For example, a repeat treatment can be performed when the patient begins to experience headache symptoms. However, preferred embodiments comprise repeating the treatment prior to the return of symptoms. Therefore, disclosed embodiments comprise repeating the treatment, for example, after 4 weeks, after 5 weeks, after 6 weeks, 8 weeks, 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment. For example, in embodiments, the frontalis can be injected in the initial treatment, then not injected in a following treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer for an osmotic effect.

A kit for practicing disclosed embodiments is also encompassed by the present disclosure. The kit can comprise a 33 gauge or smaller needle and a corresponding pen or syringe. The kit can also comprise at least one GLP-1 agonist composition. Further kits can comprise at least one GLP-1 agonist composition and at least one Clostridial neurotoxin composition, such as a botulinum type A toxin composition. The neurotoxin composition may be provided in the syringe. The kits are designed in various forms based the sizes of the syringe and the needles and the volume of the injectable composition(s) contained therein, which in turn are based on the specific deficiencies the kits are designed to treat.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Migraine

A 64-year-old female with chronic migraine has daily headaches despite ongoing treatment with anti-CGRP medications, BOTOX® injections, and antidepressants. She has a BMI of 34. She is started on a GLP-1 agonist and loses 10% of her body weight over 6 months.

Coincident with this, her mood improves, and she develops headache-free days on half the days of the month.

### Example 2

### Treatment of Migraine

A 23-year-old woman has a new onset of daily headaches associated with blurred vision. She has gained significant weight over the last 5 years while she was at college. She has a work-up completed with brain imaging studies and a spinal puncture. Her CSF pressure reading is elevated over 25 cm of CSF and she is diagnosed with headache attributed to raised intracranial pressure. She is started on a GLP-1 agonist and loses 15% of her body weight with improvement of her headaches, vision and CSF pressure.

### Example 3

### Treatment of Sleep Apnea-Associated Headache

A 45-year-old man, with a BMI of 30 and headache attributed to sleep apnea starts treatment with a GLP-1 agonist. He notes weight loss, associated with headache improvement.

### Example 4

### Treatment of Chronic Migraine

A CM patient is treated via weekly injection of 1 mg of a GLP-1 agonist.

The patient reports fewer migraines following the treatment.

### Example 5

### Treatment of Chronic Migraine

A CM patient is treated via daily injection of 0.1 mg of a GLP-1 agonist.

The patient reports fewer migraines following the treatment.

### Example 6

### Treatment of Chronic Migraine

A CM patient is treated via daily injection of 0.1 mg of a GLP-1 agonist combined with injection of 25 U of botulinum type A each at several locations along the trigeminal nerve, including the corrugator, procerus, masseter, temporalis, trapezius, and occipitalis. The frontalis and the cervical paraspinal are not injected.

The patient reports fewer migraines following the treatment.

### Example 7

### Treatment of Episodic Migraine

An EM patient is treated via daily injection of 0.05 mg of a GLP-1 agonist combined with injection of botulinum type A each at several locations along the trigeminal nerve, including the frontalis, procerus, and occipitalis. The corrugator is not injected.

The patient reports fewer migraine symptoms following the treatment.

### Example 8

### Treatment of Tension Headache

A tension headache patient is treated via daily injection of 0.1 mg of a GLP-1 agonist. The patient reports fewer migraines following the treatment.

### Example 9

### Treatment of Tension Headache

A tension headache patient is treated via daily inhalation of 1 mg of a GLP-1 agonist combined with injection of 15 U of botulinum type A each at several locations along the trigeminal nerve, including the frontalis, procerus, and occipitalis. The corrugator is not injected.

The patient reports fewer migraine symptoms following the treatment.

### Example 10

### Treatment of Cluster Headache

A cluster headache patient is treated via twice daily injection of 0.1 mg of a GLP-1 agonist. The patient reports fewer headaches following the treatment.

### Example 11

### Treatment of Cluster Headache

A cluster headache patient is treated via weekly inhalation of 2 mg of a GLP-1 agonist combined with injection of botulinum type A each at several locations along the trigeminal nerve, including the frontalis, procerus, and occipitalis. The corrugator is not injected.

The patient reports fewer symptoms following the treatment.

### Example 12

### Treatment of Trauma-Associated Headache

A trauma-associated headache patient is treated via daily injection of 0.5 mg of a GLP-1 agonist.

The patient reports fewer headaches following the treatment.

### Example 13

### Treatment of Trauma-Associated Headache

A trauma-associated headache patient is treated via weekly injection of 2 mg of a GLP-1 agonist combined with injection of botulinum type A each at several locations along the trigeminal nerve, including the frontalis, procerus, and occipitalis. The corrugator is not injected.

The patient reports fewer headache symptoms following the treatment.

### Example 14

### Treatment of Sleep Apnea-Associated Headache

A sleep apnea-associated headache patient is treated via weekly injection of 0.5 mg of a GLP-1 agonist combined with injection of 15 U of botulinum type A each at several locations along the trigeminal nerve, including the frontalis, procerus, and occipitalis.

The patient reports fewer symptoms following the treatment.

### Example 15

### Treatment of Raised Cranial Pressure-Associated Headache

A raised cranial pressure-associated headache patient is treated via weekly injection of 2 mg of a GLP-1 agonist.

The patient reports fewer symptoms following the treatment.

### Example 16

### Treatment of Raised Cranial Pressure-Associated Headache

A raised cranial pressure-associated headache patient is treated via weekly injection of 2 mg of a GLP-1 agonist combined with injection of botulinum type A each at several locations along the trigeminal nerve, including the frontalis, procerus, and occipitalis.

The patient reports fewer symptoms following the treatment.

### Example 17

### Treatment of Chronic Migraine

A CM patient is treated via the following protocol: 1 mg of a GLP-1 agonist is administered systemically. The trapezius is injected with 5 units botulinum type A per injection at two sites (superior) and 10 units per injection at two sites (inferior) on each side of the head as described in Table 1. The occipitalis is injected with 10 units per site at three sites on each side of the head, while the temporalis is injected with 5 units per site at three sites anterior to the tragus on each side of the head.

The procerus is injected at one site with 5 units. The corrugator is injected with 5 units per site at two sites, one on each side of the head. The oculi is injected with 5 units per site at two sites, one on each side of the head.

The patient reports fewer migraines for 12 weeks following the treatment.

### Example 18

### Treatment of Episodic Migraine

A migraine patient with EM is treated via the following protocol: 2 mg of a GLP-1 agonist is administered systemically. The trapezius is injected with 4 units botulinum type A per injection at two sites (superior) and 10 units per injection at two sites (inferior) on each side of the head as described in Table 1. The occipitalis is injected with 11 units per site at three sites on each side of the head, while the temporalis is injected with 4 units per site at three sites anterior to the tragus on each side of the head.

The procerus is injected at one site with 6 units. The corrugator is injected with 7 units per site at two sites, one on each side of the head. The oculi is injected with 4 units per site at two sites, one on each side of the head.

The patient reports fewer migraines for 12 weeks following the treatment.

### Example 19

### Treatment of Chronic Migraine

A migraine patient with CM is treated via the following protocol: 1 mg of a GLP-1 agonist is administered systemically. The trapezius is injected with 5 units botulinum type B per injection at two sites (superior) and 11 units per injection at two sites (inferior) on each side of the head as described in Table 1. The occipitalis is injected with 9 units per site at three sites on each side of the head, while the temporalis is injected with 4 units per site at three sites anterior to the tragus on each side of the head.

The procerus is injected at one site with 10 units. The corrugator is injected with 4 units per site at two sites, one on each side of the head. The oculi is injected with 7 units per site at two sites, one on each side of the head.

The patient reports fewer migraines for 16 weeks following the treatment.

### Example 20

### Treatment of Episodic Migraine

A migraine patient with EM is treated via the following protocol: 0.5 mg of a GLP-1 agonist is administered systemically. The trapezius is injected with 5 units botulinum type A per injection at two sites (superior) and 10 units per injection at two sites (inferior) on each side of the head as described in Table 2 and the following paragraphs. The occipitalis is injected with 10 units per site at three sites on each side of the head, while the temporalis is injected with 5 units per site at three sites anterior to the tragus on each side of the head.

The procerus is injected at one site with 5 units. The corrugator is injected with 5 units per site at two sites, one on each side of the head. The oculi is injected with 5 units per site at two sites, one on each side of the head.

The patient reports fewer migraines for 16 weeks following the treatment.

### Example 21

### Treatment of Chronic Migraine

A migraine patient with CM is treated via the following protocol: 1.8 mg of a GLP-1 agonist is administered systemically. The trapezius is injected with 5 units botulinum type A per injection at two sites (superior) and 10 units per injection at two sites (inferior) on each side of the head as described in Table 2 and the following paragraphs. The occipitalis is injected with 9 units per site at three sites on each side of the head, while the temporalis is injected with 5 units per site at three sites anterior to the tragus on each side of the head.

The procerus is injected at one site with 6 units. The corrugator is injected with 6 units per site at two sites, one on each side of the head. The oculi is injected with 4 units per site at two sites, one on each side of the head.

The patient reports fewer migraines for 13 weeks following the treatment.

### Example 22

### Treatment of Episodic Migraine

A migraine patient with EM is treated via the following protocol: 0.2 mg of a GLP-1 agonist is administered systemically. The trapezius is injected with 6 units botulinum type A per injection at two sites (superior) and 10 units per injection at two sites (inferior) on each side of the head as described in Table 2 and the following paragraphs. The occipitalis is injected with 11 units per site at three sites on each side of the head, while the temporalis is injected with 6 units per site at three sites anterior to the tragus on each side of the head.

The procerus is injected at one site with 7 units. The corrugator is injected with 5 units per site at two sites, one on each side of the head. The oculi is injected with 8 units per site at two sites, one on each side of the head.

The patient reports fewer migraines for 18 weeks following the treatment.

### Example 23

### Treatment of Episodic Migraine

A migraine patient with EM is treated via the following protocol: 3 mg of a GLP-1 agonist is administered systemically. The trapezius is injected with botulinum type A per injection at two sites (5 units superior and 10 units inferior for a total of 10 units superior and 20 units inferior) on each side of the head as described in Table 3 and the following paragraphs. The occipitalis is injected with 10 units per site at three sites on each side of the head, while the temporalis is injected with 10 units per site at three sites anterior to the tragus on each side of the head.

The procerus is injected at one site with 8 units. The corrugator is injected with 6 units per site at two sites, one on each side of the head. The oculi is injected with 5 units per site at two sites, one on each side of the head.

The patient reports fewer migraines for 14 weeks following the treatment.

### Example 24

### Treatment of Chronic Migraine

A migraine patient with CM is treated via the following protocol: 1.2 mg of a GLP-1 agonist is administered systemically. The trapezius is injected with botulinum type B per injection at two sites (6 units superior and 9 units inferior for a total of 12 units superior and 18 units inferior) on each side of the head as described in Table 3 and the following paragraphs. The occipitalis is injected with 9 units per site at three sites on each side of the head, while the temporalis is injected with 10 units per site at three sites anterior to the tragus on each side of the head.

The procerus is injected at one site with 5 units. The corrugator is injected with 5 units per site at two sites, one on each side of the head. The oculi is injected with 6 units per site at two sites, one on each side of the head.

The patient reports fewer migraines for 10 weeks following the treatment.

### Example 25

### Treatment of Chronic Migraine

A migraine patient with CM is treated via the following protocol: 1 mg of a GLP-1 agonist is administered systemically. The trapezius is injected with botulinum type A per injection at two sites (5 units superior and 10 units inferior for a total of 10 units superior and 20 units inferior) on each side of the head as described in Table 3 and the following paragraphs. The occipitalis is injected with 10 units per site at three sites on each side of the head, while the temporalis is injected with 10 units per site at three sites anterior to the tragus on each side of the head.

The procerus is injected at one site with 6 units. The corrugator is injected with 6 units per site at two sites, one on each side of the head. The oculi is injected with 5 units per site at two sites, one on each side of the head.

The nasalis is injected 2.5 units at one site on each side of the head, and the masseter is injected with 5 units at one site on each side of the head.

The patient reports fewer migraines for 12 weeks following the treatment.

### Example 26

### Treatment of Chronic Migraine

A migraine patient with CM is treated via the following protocol: 0.8 mg of a GLP-1 agonist is administered systemically. The trapezius is injected with botulinum type A per injection at two sites (5 units superior and 10 units inferior for a total of 10 units superior and 20 units inferior) on each side of the head as described in Table 4 and the following paragraphs. The occipitalis is injected with 10 units per site at three sites on each side of the head, while the temporalis is injected with 10 units per site at three sites anterior to the tragus on each side of the head.

The procerus is injected at one site with 5 units. The corrugator is injected with 5 units per site at two sites, one on each side of the head. The oculi is injected with 5 units per site at two sites, one on each side of the head.

The nasalis is injected 2.5 units at one site on each side of the head, and the masseter is injected with 5 units at one site on each side of the head.

The patient reports fewer migraines for 17 weeks following the treatment.

### Example 27

### Treatment of Migraine

A migraine patient is treated via the following protocol: 0.1 mg of a GLP-1 agonist is administered systemically. The trapezius is injected with botulinum type A per injection at two sites (5 units superior and 10 units inferior for a total of 10 units superior and 20 units inferior) on each side of the head as described in Table 4 and the following paragraphs. The occipitalis is injected with 11 units per site at three sites on each side of the head, while the temporalis is injected with 10 units per site at three sites anterior to the tragus on each side of the head.

The procerus is injected at one site with 4 units. The corrugator is injected with 6 units per site at two sites, one on each side of the head. The oculi is injected with 5 units per site at two sites, one on each side of the head.

The nasalis is injected 2.5 units at one site on each side of the head, and the masseter is injected with 5 units at one site on each side of the head.

The patient reports fewer migraines for 18 weeks following the treatment.

### Example 28

### Treatment of Migraine

A migraine patient is treated via the following protocol: 1 mg of a GLP-1 agonist is administered systemically. The trapezius is injected with botulinum type E per injection at two sites (5 units superior and 10 units inferior for a total of 10 units superior and 20 units inferior) on each side of the head as described in Table 4 and the following paragraphs. The occipitalis is injected with 9 units per site at three sites on each side of the head, while the temporalis is injected with 10 units per site at three sites anterior to the tragus on each side of the head.

The procerus is injected at one site with 6 units. The corrugator is injected with 6 units per site at two sites, one on each side of the head. The oculi is injected with 5 units per site at two sites, one on each side of the head.

The nasalis is injected 2.5 units at one site on each side of the head, and the masseter is injected with 5 units at one site on each side of the head.

The patient reports fewer migraines for 12 weeks following the treatment.

### Example 29

### Treatment of Migraine

A migraine patient is treated with botulinum type A via the following injection protocol: 1 mg of a GLP-1 agonist is administered systemically. The frontalis is injected at or near the hairline with a 5 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear threading approach injections, one 5 unit IM injection in each side of the medial corrugator muscle. The needle is inserted at the medial edge of the corrugator muscle (at the vertical crease line) and parallel to the orbital ridge horizontal plane (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 5 units per site at three sites anterior to the tragus on each side of the head by injecting the most anterior of the three sites at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear threading approach). The injection is directed laterally for first half of the dose and medially for the second half of the dose. The “linear threading” approach should allow for diffusion into the occipitalis area and greater and lesser occipital nerves

The trapezius is injected at two sites (5 units superior and 10 units inferior for a total of 10 units superior and 20 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear threading approach). For the inferior trapezius, the injection is made at a 45-degree angle at the top of the slope of the trapezius. The “linear threading” approach allows for diffusion into the superior trapezius area and 3^{rd} occipital nerve

### Example 30

### Treatment of Migraine

A migraine patient is treated with botulinum type A via the following injection protocol: 1 mg of a GLP-1 agonist is administered systemically. The frontalis is injected at or near the hairline with a 6 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear threading approach injections, one 6 unit IM injection in each side of the medial corrugator muscle. The needle is inserted at the medial edge of the corrugator muscle (at the vertical crease line) and parallel to the orbital ridge horizontal plane (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 4 units per site at two sites, one on each side of the head made at a 45-degree angle to the face with only the bevel inserted.

The temporalis is injected with 7 units per site at three sites anterior to the tragus on each side of the head by injecting the most anterior of the three sites at the hairline and angling the needle back towards the hairline of the temporal area to avoid the antra-temporal fasci.

The occipitalis is injected with 9 units per site at three sites on each side of the head, by inserting the needle parallel to the nuchal ridge. The plunger is depressed as the needle is withdrawn towards the injection site (parallel linear threading approach). The injection is directed laterally for first half of the dose and medially for the second half of the dose. The “linear threading” approach should allow for diffusion into the occipitalis area and greater and lesser occipital nerves

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear threading approach). For the inferior trapezius, the injection is made at a 45-degree angle at the top of the slope of the trapezius. The “linear threading” approach allows for diffusion into the superior trapezius area and 3^{rd} occipital nerve

### Example 31

### Treatment of Migraine

A migraine patient is treated with botulinum type B via the following injection protocol: 1.3 mg of a GLP-1 agonist is administered systemically. The frontalis is injected at the hairline with a 5 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 5 unit IM injection in each side of medial corrugator muscle. The needle is inserted at the medial edge of the corrugator muscle (at the vertical crease line) and parallel to the orbital ridge horizontal plain (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve

### Example 32

### Treatment of Migraine

A migraine patient is treated with botulinum type E via the following injection protocol: 0.3 mg of a GLP-1 agonist is administered systemically. The frontalis is injected at the hairline with a 7 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 7 unit IM injection in each side of medial corrugator muscle. The needle is inserted at the medial edge of the corrugator muscle (at the vertical crease line) and parallel to the orbital ridge horizontal plain (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 4 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves

The trapezius is injected at two sites (5 units superior and 10 units inferior for a total of 10 units superior and 20 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve

### Example 33

### Treatment of Migraine

A migraine patient is treated with botulinum type B via the following injection protocol: 1 mg of a GLP-1 agonist is administered systemically. The frontalis is injected at the hairline with a 7 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 5 unit IM injection in each side of medial corrugator muscle. The needle is inserted at the medial edge of the corrugator muscle (at the vertical crease line) and parallel to the orbital ridge horizontal plain (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 8 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve

### Example 34

### Treatment of Migraine

A migraine patient is treated with botulinum type A via the following injection protocol: 2.1 mg of a GLP-1 agonist is administered systemically. The frontalis is injected at or near the hairline with a 5 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear threading approach injections, one 5 unit IM injection in each side of the medial corrugator muscle. The needle is inserted at the medial edge of the corrugator muscle (at the vertical crease line) and parallel to the orbital ridge horizontal plane (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 10 units per site at three sites anterior to the tragus on each side of the head by injecting the most anterior of the three sites at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear threading approach). The injection is directed laterally for first half of the dose and medially for the second half of the dose. The “linear threading” approach should allow for diffusion into the occipitalis area and greater and lesser occipital nerves

The trapezius is injected at two sites (5 units superior and 10 units inferior for a total of 10 units superior and 20 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear threading approach). For the inferior trapezius, the injection is made at a 45-degree angle at the top of the slope of the trapezius. The “linear threading” approach allows for diffusion into the superior trapezius area and 3^{rd} occipital nerve

The nasalis is injected with 2.5 units at one site on each side of the head.

The masseter is injected with 5 units at one site on each side of the head.

### Example 35

### Treatment of Migraine

A migraine patient is treated with botulinum type A via the following injection protocol: 1 mg of a GLP-1 agonist is administered systemically. The frontalis is injected at or near the hairline with a 6 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear threading approach injections, one 6 unit IM injection in each side of the medial corrugator muscle. The needle is inserted at the medial edge of the corrugator muscle (at the vertical crease line) and parallel to the orbital ridge horizontal plane (parallel to the muscle above the eyebrow) and perpendicular to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 4 units per site at two sites, one on each side of the head made at a 45-degree angle to the face with only the bevel inserted.

The temporalis is injected with 7 units per site at three sites anterior to the tragus on each side of the head by injecting the most anterior of the three sites at the hairline and angling the needle back towards the hairline of the temporal area to avoid the antra-temporal fasci.

The occipitalis is injected with 9 units per site at three sites on each side of the head, by inserting the needle parallel to the nuchal ridge. The plunger is depressed as the needle is withdrawn towards the injection site (parallel linear threading approach). The injection is directed laterally for first half of the dose and medially for the second half of the dose. The “linear threading” approach should allow for diffusion into the occipitalis area and greater and lesser occipital nerves

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear threading approach). For the inferior trapezius, the injection is made at a 45-degree angle at the top of the slope of the trapezius. The “linear threading” approach allows for diffusion into the superior trapezius area and 3^{rd} occipital nerve.

The nasalis is injected with 3 units at one site on each side of the head.

The masseter is injected with 4 units at one site on each side of the head.

### Example 36

### Treatment of Migraine

A migraine patient is treated with botulinum type B via the following injection protocol: 2 mg of a GLP-1 agonist is administered systemically. The frontalis is injected at the hairline with a 5 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 5 unit IM injection in each side of medial corrugator muscle. The needle is inserted at the medial edge of the corrugator muscle (at the vertical crease line) and parallel to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve.

The nasalis is injected with 2.5 units at one site on each side of the head.

The masseter is injected with 6 units at one site on each side of the head.

### Example 37

### Treatment of Migraine

A migraine patient is treated with botulinum type E via the following injection protocol: 0.7 mg of a GLP-1 agonist is administered systemically. The frontalis is injected at the hairline with a 7 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 7 unit IM injection in each side of medial corrugator muscle. The needle is inserted at the medial edge of the corrugator muscle (at the vertical crease line) and parallel to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 4 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 10 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves

The trapezius is injected at two sites (5 units superior and 10 units inferior for a total of 10 units superior and 20 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve.

The nasalis is injected with 3.5 units at one site on each side of the head.

The masseter is injected with 5 units at one site on each side of the head.

### Example 38

### Treatment of Migraine

A migraine patient is treated with botulinum type B via the following injection protocol: 1 mg of a GLP-1 agonist is administered systemically. The frontalis is injected at the hairline with a 7 unit injection on each side of the head, with a lateral injection starting at the limbic line where the forehead starts to angle backwards, injecting at a 45-degree angle, angling away from the lower forehead.

The corrugator is then injected near the medial line, utilizing 2 parallel linear thread approach injections, one 5 unit IM injection in each side of medial corrugator muscle. The needle is inserted at the medial edge of the corrugator muscle (at the vertical crease line) and parallel to the corrugator skin line. The skin is slightly angled to skin coming in on the medial edge and the needle is injected ½″ while holding the muscle. The plunger is depressed as the needle is withdrawn towards the injection site and stopped at the medial line.

The oculi is injected with 5 units per site at two sites, one on each side of the head made at a 45-degree angle to the face with only the bevel inserted.

The temporalis is injected with 8 units per site at three sites anterior to the tragus on each side of the head by injecting at the hairline and angling the needle back towards the hairline of the temporal area to avoid the antra-temporal fasci.

The occipitalis is injected with 8 units per site at three sites on each side of the head, by inserting the needle parallel to the nuchal ridge. The plunger is depressed as the needle is withdrawn towards the injection site (parallel linear thread approach). The injection is directed laterally for first half of the dose and medially for the second half of the dose. The “linear threading” approach should allow for diffusion into the occipitalis area and greater and lesser occipital nerves.

The trapezius is injected at two sites (6 units superior and 9 units inferior for a total of 12 units superior and 18 units inferior) on each side of the head as described in Table 5 and the following paragraphs. The patient is injected at a 45-degree angle away from the mid-neck for the superior trapezius injection and parallel to C2. The plunger is depressed as the needle is withdrawn towards the injection site (parallel linear thread approach). For the inferior trapezius, the injection is made at a 45-degree angle at the top of the slope of the trapezius. The “linear threading” approach allows for diffusion into the superior trapezius area and 3^{rd} occipital nerve.

In embodiments, the nasalis is injected with 2 units at one site on each side of the head.

In embodiments, the masseter is injected with 6 units at one site on each side of the head.

### Example 39

### Treatment of Migraine

A migraine patient is treated with a GLP-1 agonist and botulinum type A in accordance with the injection protocols of Table 5 or Table 6, using the following syringe allocation for each of the injection sites specified in Table 5 or Table 6:
a. Anterior/lateral injections comprise the following:
   i. Syringe 1—32 G: injections 1 & 2 to the oculi for a total of 2 injections;
   ii. Syringe 2—32 G: injections 1 & 2 to the corrugator, injections 3 and 4 to the frontalis for a total of 4 injections;
   iii. Syringe 3—32 G: injections 1 & 2 to the nasalis for a total of 2 injections;
   iv. Syringe 4—30 G: injections 1 and 2 to the masseter for a total of 2 injections;
   v. Syringe 5—32 G: injections 1-3 to the right side of the temporalis for a total of 3 injections;
   vi. Syringe 6—32 G: injections 1-3 to the left side of the temporalis for a total of 3 injections;
b. Posterior injections comprise the following:
   i. Syringe 7—32 G: injections 1-3 to the right side of the occipitalis (since fanning) for a total of 3 injections;
   ii. Syringe 8—32 G: injections 1-3 to the left side of the occipitalis (since fanning) for a total of 3 injections;
   iii. Syringe 9—32 G: injections 1 & 2 to the superior trapezius, injections 3 & 4 to the inferior trapezius, for a total of 4 injections.

### Example 40

### Treatment of Migraine

A migraine patient is treated with a GLP-1 agonist and botulinum type A in accordance with the injection protocols of Table 5 or Table 6, using the following syringe allocation for each of the injection sites specified in Table 5 or Table 6:
a. Anterior/lateral injections comprise the following:
   i. Syringe 1—32 G: injections 1 & 2 to the oculi for a total of 2 injections;
   ii. Syringe 2—32 G: injections 1 & 2 to the corrugator, injections 3 and 4 to the frontalis for a total of 4 injections;
   iii. Syringe 3—32 G: injections 1 & 2 to the nasalis, injections 3 and 4 to the masseter for a total of 4 injections;
   iv. Syringe 5—32 G: injections 1-3 to the right side of the temporalis for a total of 3 injections;
   v. Syringe 6—32 G: injections 1-3 to the left side of the temporalis for a total of 3 injections;
b. Posterior injections comprise the following:
   i. Syringe 7—32 G: injections 1-3 to the right side of the occipitalis (since fanning) for a total of 3 injections;
   ii. Syringe 8—32 G: injections 1-3 to the left side of the occipitalis (since fanning) for a total of 3 injections;
   iii. Syringe 9—32 G: injections 1 & 2 to the superior trapezius, injections 3 & 4 to the inferior trapezius, for a total of 4 injections.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method for treating a headache disorder, the method comprising administering a GLP-1 agonist.

2. The method of claim 1, wherein said headache disorder comprises migraine, cluster, post-traumatic headache, headache attributed to sleep apnea, or headache attributed to raised intracranial pressure.

3. The method of claim 2, wherein said headache disorder comprises migraine and said migraine is associated with depression, anxiety, chronic pain syndrome, or obesity.

4. The method of claim 1, wherein the GLP-1 agonist is administered by injection, subcutaneously, trans-dermally, intradermally, systemically, or pulmonarily.

5. The method of any preceding claim, wherein said method further comprises administration of a neurotoxin.

6. The method of claim 5, wherein said administration of a neurotoxin comprises:
a) administering about 10 units of a botulinum neurotoxin into the upper frontalis at the hairline and further administering the neurotoxin to at least five of the muscle regions selected from the corrugator, masseter, nasalis, oculi, occipitalis, temporalis, and trapezius muscle regions, and wherein the cervical paraspinal muscle and procerus muscles are not administered-to;
b) thereby treating the migraine headache.

7. The method of claim 6, wherein said botulinum neurotoxin is further administered to between five and seven of the muscle regions selected from the corrugator, masseter, nasalis, occipitalis, temporalis, trapezius and oculi muscle regions.

8. The method of claim 6, wherein said botulinum neurotoxin is further administered to the corrugator, occipitalis, temporalis, and trapezius muscle regions.

9. The method of claim 6, wherein said botulinum neurotoxin is further administered to the corrugator, occipitalis, temporalis, oculi and trapezius muscle regions.

10. The method of claim 6, wherein said botulinum neurotoxin is further administered to the corrugator, occipitalis, temporalis, oculi, masseter and trapezius muscle regions.

11. The method of claim 6, wherein said botulinum neurotoxin is further administered to the corrugator, occipitalis, temporalis, oculi, nasalis, and trapezius muscle regions.

12. The method of claim 6, wherein said botulinum neurotoxin is further administered to the corrugator, occipitalis, temporalis, oculi, nasalis, masseter and trapezius muscle regions.

13. The method of claim 6, wherein said migraine headache comprises an episodic migraine or a chronic migraine.

14. The method of claim 13, wherein said migraine headache comprises an episodic migraine.

15. The method of claim 13, wherein said migraine headache comprises a chronic migraine.

16. The method of claim 6, wherein said botulinum neurotoxin comprises botulinum type A.

17. The method of claim 6, wherein said botulinum neurotoxin comprises botulinum type B, C, D, E, or F.

18. The method of claim 6, wherein the total dose of said botulinum neurotoxin comprises about 145-200 Units.

19. The method of claim 18, wherein the total dose of said botulinum neurotoxin comprises about 195 Units.

20. The method of claim 18, wherein the total dose of said botulinum neurotoxin comprises about 150 Units.

21. The method of claim 6, wherein said administration to said corrugator comprises administration of said botulinum neurotoxin in an amount of about 10 Units.

22. The method of claim 6, wherein said administration to said oculi comprises administration of said botulinum neurotoxin in an amount of about 10 Units.

23. The method of claim 6, wherein said administration to said temporalis comprises administration of said botulinum neurotoxin in an amount of about 30 Units anterior to the tragus.

24. The method of claim 6, wherein said administration to said temporalis comprises administration of said botulinum neurotoxin in an amount of about 60 Units anterior to the tragus.

25. The method of claim 6, wherein said administration to said nasalis comprises administration of said botulinum neurotoxin in an amount of about 5 Units.

26. The method of claim 6, wherein said administration to said masseter comprises administration of said botulinum neurotoxin in an amount of about 10 Units.

27. The method of claim 6, wherein said administration to said occipitalis comprises administration of said botulinum neurotoxin in an amount of about 60 Units.

28. The method of claim 6, wherein said administration to said trapezius comprises administration of said botulinum neurotoxin in an amount of about 30 Units.

29. The method of claim 28, wherein said administration to said trapezius comprises superficial administration of said botulinum neurotoxin in an amount of about 10 Units to the superior trapezius.

30. The method of claim 6, wherein said nasalis is not administered-to.

31. The method of claim 6, wherein said masseter is not administered-to.

32. A fixed-site, fixed-dose method for treating migraine with a GLP-1 agonist and 22 individual botulinum administrations comprising:
administering the GLP-1 agonist; and
administering a botulinum toxin to the superior trapezius at a dosage of about 5 units per site at one site on each side of the head and about 10 units per site to the inferior trapezius at one site on each side of the head; and
administering a botulinum toxin to the occipitalis at a dosage of about 10 units per site at three sites on each side of the head; and
administering a botulinum toxin to the temporalis at a dosage of about 5 units per site at three sites on each side of the head; and
administering a botulinum toxin to the oculi at a dosage of about 5 units to one site on each side of the head; and
administering a botulinum toxin to the corrugator at a dosage of about 5 units to one site on each side of the head; and
administering a botulinum toxin to the upper frontalis at the hairline at a dosage of about 5 units to one site on each side of the head;
wherein the botulinum toxin is not administered to the cervical paraspinal or procerus muscles;
thereby treating the migraine with a total of about 150 Units.

33. A fixed-site, fixed-dose method for treating migraine with a GLP-1 agonist and 26 individual botulinum administrations comprising:
administering the GLP-1 agonist; and
administering a botulinum toxin to the superior trapezius at a dosage of about 5 units per site at one site on each side of the head and about 10 units per site to the inferior trapezius at one site on each side of the head; and
administering a botulinum toxin to the occipitalis at a dosage of about 10 units per site at three sites on each side of the head; and
administering a botulinum toxin to the temporalis at a dosage of about 10 units per site at three sites on each side of the head; and
administering a botulinum toxin to the oculi at a dosage of about 5 units to one site on each side of the head; and
administering a botulinum toxin to the corrugator at a dosage of about 5 units to one site on each side of the head; and
administering a botulinum toxin to the upper frontalis at the hairline at a dosage of about 5 units to one site on each side of the head; and
administering a botulinum toxin to the nasalis at a dosage of about 2.5 units to one site on each side of the head; and
administering a botulinum toxin to the masseter at a dosage of about 5 units to one site on each side of the head;
wherein the botulinum toxin is not administered to the cervical paraspinal or procerus muscles;
thereby treating the migraine with a total of about 195 Units.

34. A fixed site method for treating migraine headache with a GLP-1 agonist and 24 individual botulinum administrations comprising:
administering the GLP-1 agonist; and
administering a botulinum toxin to the superior trapezius at a dosage of about 3-10 units per site at one site on each side of the head and 5-15 units per site to the inferior trapezius at one site on each side of the head; and
administering a botulinum toxin to the occipitalis at a dosage of about 7-15 units per site at three sites on each side of the head; and
administering a botulinum toxin to the temporalis at a dosage of about 3-15 units per site at three sites on each side of the head; and
administering a botulinum toxin to the oculi at a dosage of about 3-10 units to one site on each side of the head; and
administering a botulinum toxin to the corrugator at a dosage of about 3-10 units to one site on each side of the head; and
administering a botulinum toxin to the upper frontalis at the hairline at a dosage of about 3-10 units to one site on each side of the head; and
administering a botulinum toxin to at least one of the nasalis and masseter, wherein said nasalis administration is at a dosage of about 1-5 units to one site on each side of the head; and said masseter administration is at a dosage of about 3-10 units to one site on each side of the head;
wherein the botulinum toxin is not administered to the cervical paraspinal or procerus muscles;
thereby treating the migraine headache.

35. A method of reducing the amount of neurotoxin used for treating a migraine comprising substituting a GLP-1 agonist for at least part of the neurotoxin dosage.

36. A method of increasing the duration of a neurotoxin-based migraine treatment by supplementing the neurotoxin administration with a GLP-1 agonist.
